(19) United States
(12) Patent Application Publication
(10) Pub. No.: US 2025/0177516 A1
(43) Pub. Date: Jun. 5, 2025

(54) METHODS FOR PRODUCING AN ADJUVANT

(71) Applicant: Pfizer Inc., New York, NY (US)

(72) Inventors: Parisa Ansari, Chelmsford, MA (US); Advait Vijay Badkar, Westford, MA (US); Leena Shriram Bagle, Edgewater, NJ (US); Zoltan Beck, Rockville, MD (US); Bo Chen, Ridgewood, NJ (US); Ramin Darvari, Lexington, MA (US); Parag Ashok Kolhe, Westford, MA (US); Maria Sergeyevna Lapshina, Harrison, NJ (US); Rachna Gopalkrishna Nayak, Rockville, MD (US); Naveen Palath, Westford, MA (US); Shuai Shi, Whippany, NJ (US)

(21) Appl. No.: 18/845,182

(22) PCT Filed: Mar. 9, 2023
(86) PCT No.: PCT/IB2023/052255

§ 371 Date: Sep. 9, 2024

Related U.S. Application Data
(60) Provisional application No. 63/485,964, filed on Feb. 20, 2023.
(60) Provisional application No. 63/319,418, filed on Mar. 14, 2022.

(51) Int. Cl. A61K 39/39; A61K 9/1277; A61K 39/00

(52) U.S. Cl. CPC A61K 39/39; A61K 9/1277; A61K 2039/55555; A61K 2039/55572; A61K 2039/55577

(57) ABSTRACT

This invention provides methods for producing homogeneous and heterogeneous adjuvant formulations comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol wherein the mole percent concentration of the cholesterol in the liposome composition is greater than 50% (mol/mol), and further provides the adjuvant formulations produced from said methods.

### CROSS REFERENCE TO RELATED APPLICATIONS

This application claims the benefits of U.S. Provisional Application No. 63/485,964 filed Feb. 20, 2023, and U.S. Provisional Application No. 63/319,418 filed Mar. 14, 2022. The entire content of each of the foregoing applications is incorporated herein by reference.

### FIELD OF THE INVENTION

This invention provides methods for producing homogeneous and heterogeneous adjuvant formulations comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol), and further provides the adjuvant formulations produced from said methods.

### BACKGROUND OF THE INVENTION

A commercial liposomal adjuvant formulation, known as AS01, containing both monophosphoryl lipid A (MPLA) and QS-21 saponin, was used as the adjuvant for a Shingles vaccine in 2017 in the United States for adults>50 years of age. The US Army has developed another liposome-based formulation containing both MPLA and QS-21 saponin, which is known as ALFQ (Army Liposome Formulation Q). ALFQ has been shown to be potent as a liposomal vaccine adjuvant in rodents and non-human primate studies, and it has also been shown to be non-pyrogenic and nontoxic in preclinical studies. The improved safety profile of ALFQ can be attributed to the irreversible binding by liposomal cholesterol to free QS-21 to form a complex that prevents hemolysis resulting from the binding of QS-21 to erythrocytes. The size of ALFQ increases from 50 to 100 nm to as large as approximately 30,000 nm during the manufacture of ALFQ when soluble QS-21 is added to a suspension of the liposome intermediate. As described in the literature, the ALFQ liposome intermediate is prepared by a rehydration process (Beck et al., Biochimica et Biophysica Acta 1848 (2015) 775-780; Singh et al., Biochemical and Biophysical Research Communications 529 (2020) 362-365; Matyas et al., Methods in Enzymology 373 (2003) 34-50). In that process, lipids are mixed and dissolved in organic solvent, dried under vacuum, and then liposomes are formed in PBS and downsized by microfluidizer to 30-100 nm. In the most widely used of these methods, a thin lipid film (from an organic solvent) is deposited on the walls of a container, an aqueous solution of the material to be encapsulated is added, and the container is agitated (Bangham et al., J. Mol. Biol. 13 (1965) 238-252). Under the right conditions, this process results in the formation of multilamellar vesicles of liposomes. However, this method is not generally scalable for manufacturing, due to size limitations of the equipment used, i.e. a Rotavap.

In another process, an organic solution of lipid is freeze-dried, resulting in a lyophilized product with physical properties for easy hydration by an aqueous solution of the material to be encapsulated. Problems associated with this method of production includes variability of drug encapsulation in the liposomes between batches. For example, Conrad et al. (Biochim. Biophys. Acta 332 (1974) 36-46) shows that a standard deviation in encapsulation efficiency of 12-13% was found between independently prepared liposome preparations using this method.

Therefore, there is a need for reproducible processes that can control the size and polydispersity of liposomal adjuvant formulations containing MPLA and a saponin including, but not limited to, QS-21. Additionally, there is a need for processes that are sufficiently robust to be used at large scale, i.e. scalable for clinical and commercial manufacturing.

### SUMMARY OF THE INVENTION

This invention provides novel reproducible processes that can control the size and polydispersity of liposomal adjuvant formulations comprising MPLA and a saponin including, but not limited to, QS-21. Additionally, this invention provides processes for large scale manufacturing of said liposomal adjuvant formulations comprising MPLA and a saponin, wherein large scale manufacturing (i.e. scalable manufacturing) produces liposomal adjuvant formulations in amounts sufficient for clinical and commercial use.

This invention provides a first method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG), and a combination thereof, and (ii) cholesterol, wherein the mole percent concentration of the cholesterol in the liposome composition is greater than 50% (mol/mol), said method comprising the steps of:
(i) dissolving the phospholipids, cholesterol and MPLA in an organic solvent to form an organic phase;
(ii) injecting the organic phase of step (i) into an aqueous phase at a specific flowrate and at a specific ratio of the organic phase to the aqueous phase to form a liposome; (iii) stirring the liposome of step (ii) to form an intermediate liposome;
(iv) removing the organic phase of the intermediate liposome of step (iii);
(v) concentrating the intermediate liposome of step (iv); and
(vi) compounding the intermediate liposome of step (v) with a saponin to form a final adjuvant formulation having a size range of about 30-400 nm with a polydispersity of 0.05 to 0.5, thereby producing the homogeneous adjuvant formulation.

In one embodiment of the first method for producing a homogeneous adjuvant formulation, the saponin is selected from the group consisting of QS-7, QS-18, QS-21, or a mixture thereof. In a preferred embodiment the saponin is QS-21.

This invention provides a second method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG) and a combination thereof, and (ii) cholesterol, wherein the mole percent concentration of the cholesterol in the liposome composition is greater than 50% (mol/mol),

said method comprising the steps of:
(i) dissolving the phospholipids, cholesterol and MPLA in an organic solvent or a mixture of organic solvents to form an organic phase;
(ii) injecting the organic phase of step (i) into an aqueous phase at a specific flowrate and at a specific ratio of the organic phase to the aqueous phase to form an intermediate liposome;
(iii) concentrating the intermediate liposome of step (ii);
(iv) removing the organic phase of the intermediate liposome of step (iii);
(v) filtering the intermediate liposome of step (iv); and
(vi) compounding the intermediate liposome of step (v) with a saponin to form a final adjuvant formulation having a size range of about 30-400 nm with a polydispersity of 0.05 to 0.5, thereby producing the homogeneous adjuvant formulation.

In one embodiment of the second method for producing a homogeneous adjuvant formulation, the saponin is selected from the group consisting of QS-7, QS-18, QS-21, or a mixture thereof. In a preferred embodiment, the saponin is QS-21.

This invention provides the first method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol),

said method comprising the steps of:
(i) dissolving the phospholipids, cholesterol and MPLA in an organic solvent to form a lipid solution as an organic phase;
(ii) simultaneously injecting the organic phase of step (i) and an aqueous phase together at specific flowrates and at a specific ratio of the organic solvent phase to the aqueous phase to form a liposome;
(iii) concentrating the intermediate liposome of step (ii);
(iv) removing the organic phase of the intermediate liposome of step (iii);
(v) treating the intermediate liposome of step (iv) by microfluidizer for 10 passes at ˜17500 psi;
(vi) filtering the intermediate liposome of step (v) using 0.22 um membrane; and
(vii) compounding the intermediate liposome of step (vi) with a saponin, thereby producing the heterogeneous adjuvant formulation.

In one embodiment of the first method for producing a heterogeneous adjuvant formulation, the saponin is selected from the group consisting of QS-7, QS-18, QS-21, or a mixture thereof. In a preferred aspect, the saponin is QS-21.

This invention provides a second method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol),

said method comprising the steps of:
(i) dissolving the phospholipids, cholesterol and MPLA in an organic solvent to form a lipid formulation an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) concentrating the intermediate liposome of step (iii);
(v) removing the organic phase of the intermediate liposome of step (iv); and
(vi) compounding the intermediate liposome of step (vi) with a saponin,
   thereby producing the heterogeneous adjuvant formulation.

In one embodiment of the second method for producing a heterogeneous adjuvant formulation, the saponin is selected from the group consisting of QS-7, QS-18, QS-21, or a mixture thereof. In a preferred aspect, the saponin is QS-21.

This invention provides a third method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol),

said method comprising the steps of:
(i) preparing a lyophilized organic phase comprising the phospholipids, cholesterol and MPLA;
(ii) rehydrating the lyophilized organic phase of step (i) with an aqueous phase to form an intermediate liposome;
(iii) downsizing of the intermediate liposome of step (ii) using microfluidizer;
(iv) compounding the intermediate liposome of step (iii) with a saponin,
   thereby producing the heterogeneous adjuvant formulation.

In one embodiment of the third method for producing a heterogeneous adjuvant formulation, the saponin is selected from the group consisting of QS-7, QS-18, QS-21, or a mixture thereof. In a preferred aspect, the saponin is QS-21.

This invention also provides an adjuvant formulation produced by any one of the methods described herein.

### BRIEF DESCRIPTION OF THE DRAWINGS

FIG. 1 shows the neutralization titers of individual animals (rats) immunized with C. difficile toxoid antigens formulated with different LiNA-2 adjuvants (homogeneous and heterogeneous).

### DETAILED DESCRIPTION OF THE INVENTION

This invention relates to processes for preparing adjuvant formulations comprising a monophosphoryl lipid A (MPLA)-containing liposome composition and at least one saponin (e.g., QS-21). Examples of such non-toxic adjuvant formulations are described in U.S. Pat. No. 10,434,167 and PCT International Publication No. WO2015148648, which are each incorporated by reference herein in their respective entireties. This adjuvant formulation comprises a monophosphoryl lipid A (MPLA)-containing liposome composition and at least one saponin, wherein the liposome composition comprises i) a lipid bilayer comprising phospholipids and ii) cholesterol at a mole percent concentration of the liposome composition of greater than about 50% (mol/mol). The saponin may be selected from QS-7, QS-18, QS-21, or a mixture thereof. Preferably, the saponin is QS-21.

Hence said adjuvant formulation comprises a monophosphoryl lipid A (MPLA)-containing liposome composition and at least one saponin, wherein the liposome composition comprises i) a lipid bilayer comprising phospholipids and ii) cholesterol, where the molar ratio of cholesterol to phospholipids is greater than about 1. The saponin may be selected from QS-7, QS-18, QS-21, or a mixture thereof. Preferably, the saponin is QS-21. Multiple processes are described herein to prepare heterogeneous and homogeneous liposomal adjuvant formulations scalable for manufacturing. The homogeneous process is a controlled and robust process generating size-controlled liposomes. Both adjuvant formulations compromise a MPLA-containing liposome comprising a saponin (e.g., QS-21). The non-toxic adjuvant formulation described in U.S. Pat. No. 10,434,167 is a highly heterogeneous product manufactured by a process that is very difficult to scale-up for clinical or commercial manufacturing. Example 3 describes three novel processes to manufacture this heterogeneous liposomal adjuvant formulation. These novel processes are highly reproducible and can be easily scaled-up for clinical and commercial manufacturing.

Examples 1 and 2 describe two processes that are scalable for manufacturing homogeneous liposomal adjuvant formulations having the same components as the heterogeneous liposomal adjuvant formulation described in U.S. Pat. No. 10,434,167. The processes described by Examples 1 and 2 create a homogeneous formulation having liposomes with a size range less than 1 micrometer and a controlled polydispersity index (PDI) ranging from 0.05 to 0.3. The process described herein is reproducible and well-controlled which enables the manufacture of a homogeneous liposomal adjuvant formulation having a defined size.

Also described herein are multiple novel solvent injection methods to prepare the intermediate liposomes. Solvent injection involves quick injection of the lipid solution in ethanol or other organic solvents into an aqueous medium. The process is performed either at room temperature or at a higher temperature (e.g., 60° C.). The size of intermediate liposomes can be generated from 30 nm to 200 nm with a PDI<0.25. The size and PDI of the intermediate can be controlled by adjusting the parameters of solvent injection such as: temperature, flow rate, flow rate ratio of organic to aqueous phases, or with the treatment of a microfluidizer. The final size of the liposomal adjuvant formulation can be influenced by the size and the manufacturing process of the intermediate liposome (e.g. the number of passes by microfluidizer, etc.) as described herein.

Furthermore, the preparation of heterogeneous liposomes using freeze-dried lipids are also described herein. In this preparation there is no problem of drug loading variation in different batches as there are no drugs in the adjuvant formulation. This method ensures uniformity from batch to batch in clinical and commercial scales and facilitates quality control.

Overall, provided herein are novel processes to prepare adjuvant formulations comprising a MPLA-containing liposome composition comprising a saponin (e.g., QS-21). These processes can control the size and polydispersity of the liposomal adjuvant formulations and are easily scalable for clinical and commercial manufacturing and reproducible compared to conventional processes. In addition, provided herein is a novel homogeneous MPLA-containing liposome composition comprising a saponin (e.g. QS-21).

Accordingly, this invention provides a first method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater 50% (mol/mol), said method comprising the steps of:
(i) dissolving the phospholipids, cholesterol and MPLA in an organic solvent to form an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) removing the organic phase of the intermediate liposome of step (iii);
(v) concentrating the intermediate liposome of step (iv); and
(vi) compounding the intermediate liposome of step (v) with a saponin to form a final adjuvant formulation having a size range of about 30-400 nm with a polydispersity of 0.05 to 0.5, thereby producing the homogeneous adjuvant formulation.

In one embodiment of the first method for producing a homogeneous adjuvant composition, the saponin is selected from the group consisting of QS-7, QS-18, QS-21, or a mixture thereof. In a preferred embodiment the saponin is QS-21.

In another embodiment of the first method for producing a homogeneous adjuvant composition, wherein in step (i) the phospholipids, cholesterol and MPLA are dissolved in the organic solvent by sonication, heat or a combination thereof, preferably by heating. In one aspect, the organic solvent is ethanol or isopropyl alcohol. In another aspect, the organic phase is heated to a temperature between 45° C. to 65° C.

In another embodiment of the first method for producing a homogeneous adjuvant composition, the aqueous phase comprises water, and optionally, a buffer. In a preferred aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature between 20° C. to 60° C. In another aspect, the flowrate of step (ii) is 0.5 mL/min to 400 mL/min or quick addition. In another aspect, the flowrate of step (ii) is 0.5 mL/min to 400 mL/min. In another aspect, the injection of step (ii) is conducted by rapid injection of the organic phase of step (i).

In another embodiment of the first method for producing a homogeneous adjuvant composition, the intermediate liposome of step (iii) is stirred at a rate of 700 rpm to 900 rpm.

In another embodiment of the first method for producing a homogeneous adjuvant composition, the ratio of organic phase to aqueous phase of step (ii) ranges from 1:4 to 1:16. In a preferred embodiment, the ratio is 1:8.

In another embodiment of the first method for producing a homogeneous adjuvant composition, the size of the intermediate liposome in step (iv) is downsized by using a high pressure extruder or a microfluid homogenizer. In one aspect, the size of the liposome is downsized in step (iv) by using membrane sizes ranging from 50 nm to 120 nm or homogenization pressures between 17000 PSI and 24000 PSI, or a combination of both.

In another embodiment of the first method for producing a homogeneous adjuvant composition, the organic solvent is removed before downsizing of step (iv) or after downsizing of step (iv). In one aspect, removing the organic phase of the intermediate liposome of step (iv) is by Tangential Flow Filtration (TFF). In another aspect, the TFF is TFF diafiltration. In another aspect, the TFF comprises membranes having a molecular weight cut-off (MWCO) ranging from 100-500 kDa.

In another embodiment of the first method for producing a homogeneous adjuvant composition, the concentrating of step (v) is by Ultrafiltration. In one aspect, the Ultrafiltration comprises a bioburden reduction filter and a sterile filter.

In another embodiment of the first method for producing a homogeneous adjuvant composition, the compounding of step (vi) is at a mixing speed of 300 rpm. In one aspect, the compounding of step (vi) ranges from 1 hour to 24 hours. In another aspect, the compounding of step (vi) occurs at room temperature or from 2-8° C.

In another embodiment of the first method for producing a homogeneous adjuvant composition, the final adjuvant formulation has a size range of about 30 nm-400 nm.

In another embodiment of the first method for producing a homogeneous adjuvant composition, the final adjuvant formulation has a polydispersity of 0.05 to 0.5.

In a further embodiment of the first method for producing a homogeneous adjuvant composition, the injecting of step (ii) is by pump or syringe injection. In a preferred aspect, the injecting of step (ii) is by pump.

In an embodiment of the first method for producing a homogeneous adjuvant composition, said at least one phospholipid is a mixture of dimyristoyl phosphatidylcholine (DMPC) and dimyristoyl phosphatidylglycerol (DMPG).

In an embodiment, said first method for producing a homogeneous adjuvant formulation comprises a liposome bilayer comprising (a) monophosphoryl lipid A (MPLA), (b) a saponin, and (c) a liposome composition comprising (i) at least one phospholipid, wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the molar ratio of cholesterol to phospholipid in the liposome composition is greater than 1, comprises the steps of:
(i) dissolving the phospholipids, cholesterol and MPLA in an organic solvent to form an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) removing the organic phase of the intermediate liposome of step (iii);
(v) concentrating the intermediate liposome of step (iv); and
(vi) compounding the intermediate liposome of step (v) with a saponin to form a final adjuvant formulation having a size range of about 30-400 nm with a polydispersity of 0.05 to 0.5,
   thereby producing the homogeneous adjuvant formulation.

This invention provides a second method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol),

said method comprising the steps of:
(i) dissolving the phospholipids, cholesterol and MPLA in an organic solvent or a mixture of organic solvents to form an organic phase;
(ii) injecting the organic phase of step (i) into an aqueous phase at a specific flowrate and at a specific ratio of the organic phase to the aqueous phase to form an intermediate liposome;
(iii) concentrating the intermediate liposome of step (ii);
(iv) removing the organic phase of the intermediate liposome of step (iii);
(v) filtering the intermediate liposome of step (iv) and
(vi) compounding the intermediate liposome of step (v) with a saponin to form a final adjuvant formulation having a size range of about 30-400 nm with a polydispersity of 0.05 to 0.5,
   thereby producing the homogeneous adjuvant formulation.

In one embodiment of the second method for producing a homogeneous adjuvant composition, the saponin is selected from the group consisting of QS-7, QS-18, QS-21, or a mixture thereof. In a preferred embodiment, the saponin is QS-21.

In another embodiment of the second method for producing a homogeneous adjuvant composition, in step (i) the phospholipids, cholesterol and MPLA are dissolved in the organic solvent by sonication, heat, stirring or a combination thereof. In one aspect, the organic solvent is ethanol or isopropyl alcohol or other organic solvents. In one aspect, the organic solvent is ethanol or isopropyl alcohol. In another aspect, the organic phase is heated to a temperature between 45° C. to 65° C. Preferably, between 50° C. to 65° C. or between 45° C. to 55° C.

In another embodiment of the second method for producing a homogeneous adjuvant composition, the aqueous phase comprises water, and optionally, a buffer. In one aspect, the buffer comprises 10 mM phosphate at about pH 6.2 containing 150 mM NaCl. In a preferred aspect, the aqueous phase is 10 mM phosphate at pH 6.2 containing 150 mM NaCl. In another aspect, the aqueous phase is at a temperature between 20° C. to 65° C. In another aspect, the flowrate of step (ii) is 5 mL/min to 15 mL/min. In another aspect, the flowrate of step (ii) is 12 mL/min. In another aspect, the intermediate liposome of step (ii) is stirred at a rate of 100 rpm to 1000 rpm. In another aspect, the ratio of organic phase to aqueous phase of step (ii) ranges from 1:2 to 1:16. In a preferred embodiment, the ratio is 4:7.

In another embodiment of the second method for producing a homogeneous adjuvant composition, the concentrating of step (iii) is by Ultrafiltration.

In another embodiment of the second method for producing a homogeneous adjuvant composition, removing the organic phase of the intermediate liposome of step (iv) is by Tangential Flow Filtration (TFF). In one aspect, the TFF is TFF diafiltration. In another aspect, the TFF comprises membranes having a molecular weight cut-off (MWCO) ranging from 100-500 kDa.

In another embodiment of the second method for producing a homogeneous adjuvant composition, the filtering of step (v) comprises a bioburden reduction filter and a sterile filter.

In another embodiment of the second method for producing a homogeneous adjuvant composition, the compounding of step (vi) is at a mixing speed of 350 rpm for 1 hour at RT.

In another embodiment of the second method for producing a homogeneous adjuvant composition, the final adjuvant formulation has a size range of about 50-200 nm. Preferably, the final adjuvant formulation has a size of about 100 nm.

In another embodiment of the second method for producing a homogeneous adjuvant composition, the final adjuvant formulation has a polydispersity of 0.05 to 0.5. Preferably, the final adjuvant formulation has a PDI of <0.2

In a further embodiment of the second method for producing a homogeneous adjuvant composition, the injecting of step (ii) is by pump or syringe injection. In a preferred aspect, the injecting of step (ii) is by pump.

In an embodiment of the second method for producing a homogeneous adjuvant composition, said at least one phospholipid is a mixture of dimyristoyl phosphatidylcholine (DMPC) and dimyristoyl phosphatidylglycerol (DMPG).

In an embodiment, said second method for producing a homogeneous adjuvant formulation comprises a liposome bilayer comprising (a) monophosphoryl lipid A (MPLA), (b) a saponin, and (c) a liposome composition comprising (i) at least one phospholipid selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG) and combination thereof, and (ii) cholesterol wherein the molar ratio of cholesterol to phospholipid is greater than 1,

comprises the steps of:
(i) dissolving the phospholipids, cholesterol and MPLA in an organic solvent or a mixture of organic solvents to form an organic phase;
(ii) injecting the organic phase of step (i) into an aqueous phase at a specific flowrate and at a specific ratio of the organic phase to the aqueous phase to form an intermediate liposome;
(iii) concentrating the intermediate liposome of step (ii);
(iv) removing the organic phase of the intermediate liposome of step (iii);
(v) filtering the intermediate liposome of step (iv) and
(vi) compounding the intermediate liposome of step (v) with a saponin to form a final adjuvant formulation having a size range of about 30-400 nm with a polydispersity of 0.05 to 0.5,
   thereby producing the homogeneous adjuvant formulation.

This invention provides a first method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and combination thereof, and (ii) cholesterol, wherein the mole percent concentration of the cholesterol in the liposome composition is greater than 50% (mol/mol),

said method comprising the steps of:
(i) dissolving the phospholipids, cholesterol and MPLA in an organic solvent to form a lipid solution as an organic phase;
(ii) simultaneously injecting the organic phase of step (i) and an aqueous phase together at specific flowrates and at a specific ratio of the organic solvent phase to the aqueous phase to form a liposome;
(iii) concentrating the intermediate liposome of step (ii);
(iv) removing the organic phase of the intermediate liposome of step (iii);
(v) treating the intermediate liposome of step (iv) by microfluidizer for 10 passes at ˜17500 psi;
(vi) filtering the intermediate liposome of step (v) using 0.22 um membrane; and
(vii) compounding the intermediate liposome of step (vi) with a saponin,
   thereby producing the heterogeneous adjuvant formulation.

In one embodiment of the first method for producing a heterogeneous adjuvant formulation, the saponin is selected from the group consisting of QS-7, QS-18, QS-21, or a mixture thereof. In a preferred aspect, the saponin is QS-21.

In another embodiment of the first method for producing a heterogeneous adjuvant formulation, in step (i) the phospholipids, cholesterol and MPLA are dissolved in the organic solvent by sonication, heat, stirring or a combination thereof. In one aspect, the organic solvent is ethanol or isopropyl alcohol or their mixture. In another aspect, the lipid formulation organic phase is heated to a temperature of 45-65° C.

In another embodiment of the first method for producing a heterogeneous adjuvant formulation, the aqueous phase comprises water, and optionally, a buffer. In one aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature between 45-60° C.

In another embodiment of the first method for producing a heterogeneous adjuvant formulation, in step (ii) the organic phase has a flowrate of 0.5 to 2 ml/min and the aqueous phase has a flowrate of 5 to 15 ml/min. In another embodiment of the third method, in step (ii) the organic phase has a flowrate of 1.333 ml/min and the aqueous phase has a flowrate of 10.667 ml/min.

In another embodiment of the first method for producing a heterogeneous adjuvant formulation, the intermediate liposome of step (iii) is stirred at a rate of 100 rpm to 900 rpm.

In another embodiment of the first method for producing a heterogeneous adjuvant formulation, the ratio of organic phase to aqueous phase of step (ii) ranges from 1:4 to 1:8. In a preferred aspect, the ratio is 1:8.

In another embodiment of the first method for producing a heterogeneous adjuvant formulation, removing the organic phase of the intermediate liposome of step (iii) and step (iv) is by Tangential Flow Filtration (TFF). In one aspect, the TFF is TFF ultrafiltration and diafiltration.

In another aspect, the TFF comprises membranes having a molecular weight cut-off (MWCO) ranging from 100-500 kDa.

In another embodiment of the first method for producing a heterogeneous adjuvant formulation, the concentrating of step (iii) is by Ultrafiltration.

In another embodiment of the first method for producing a heterogeneous adjuvant formulation, the size of the intermediate liposome in step (iv) is treated by using a microfluidizer.

In another embodiment of the first method for producing a heterogeneous adjuvant formulation, the organic solvent is removed before microfluidizing of step (v).

In another embodiment of the first method for producing a heterogeneous adjuvant formulation, a buffer is added to the compounding of step (vii). In one aspect, the compounding of step (vii) is at a mixing speed of 350 rpm for 1 hour to 48 at RT or agitated for 1 hr. In another aspect, following the compounding of step (vii) the intermediate liposome is stored at RT without stirring for up to 48 hours.

In another embodiment of the first method for producing a heterogeneous adjuvant formulation, the final adjuvant formulation has a size of about 300 nm to 1000 nm.

In another embodiment of the first method for producing a heterogeneous adjuvant formulation, the final adjuvant formulation has a polydispersity of 0.4-1.0.

In a further embodiment of the first method for producing a heterogeneous adjuvant formulation, the injecting of step (ii) is by Nanoassemblr or pump or syringe injection.

In an embodiment of the first method for producing a heterogeneous adjuvant formulation, said at least one phospholipid is a mixture of dimyristoyl phosphatidylcholine (DMPC) and dimyristoyl phosphatidylglycerol (DMPG).

In an embodiment, said first method for producing a heterogeneous adjuvant formulation comprises a liposome bilayer comprising (a) monophosphoryl lipid A (MPLA), (b) a saponin, and (c) a liposome composition comprising (i) at least one phospholipid selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG) and combination thereof, and (ii) cholesterol, wherein the molar ratio of cholesterol to phospholipid is greater than 1, comprises the steps of:
(i) dissolving the phospholipids, cholesterol and MPLA in an organic solvent to form a lipid solution as an organic phase;
(ii) simultaneously injecting the organic phase of step (i) and an aqueous phase together at specific flowrates and at a specific ratio of the organic solvent phase to the aqueous phase to form a liposome;
(iii) concentrating the intermediate liposome of step (ii);
(iv) removing the organic phase of the intermediate liposome of step (iii);
(v) treating the intermediate liposome of step (iv) by microfluidizer for 10 passes at ˜17500 psi;
(vi) filtering the intermediate liposome of step (v) using 0.22 um membrane; and
(vii) compounding the intermediate liposome of step (vi) with a saponin,
   thereby producing the heterogeneous adjuvant formulation.

This invention provides a second method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol),

said method comprising the steps of:
(i) dissolving the phospholipids, cholesterol and MPLA in an organic solvent to form a lipid formulation an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) concentrating the intermediate liposome of step (iii);
(v) removing the organic phase of the intermediate liposome of step (iv); and
(vi) compounding the intermediate liposome of step (vi) with a saponin,
   thereby producing the heterogeneous adjuvant formulation.

In one embodiment of the second method for producing a heterogeneous adjuvant formulation, the saponin is selected from the group consisting of QS-7, QS-18, QS-21, or a mixture thereof. In a preferred aspect, the saponin is QS-21.

In another embodiment of the second method for producing a heterogeneous adjuvant formulation, in step (i) the phospholipids, cholesterol and MPLA are dissolved in the organic solvent by sonication, heat, stirring or a combination thereof. In one aspect, the organic solvent comprises ethyl acetate and isopropyl alcohol. In another aspect, the lipid formulation organic phase is heated to a temperature from 50° C. to 65° C.

In another embodiment of the second method for producing a heterogeneous adjuvant formulation, the aqueous phase comprises water, and optionally, a buffer. In one aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature of 20-25° C. In a further aspect, the flowrate of step (ii) is 10 to 30 mL/min. In a further aspect, the flowrate of step (ii) is 20 mL/min.

In another embodiment of the second method for producing a heterogeneous adjuvant formulation, the intermediate liposome of step (iii) is stirred at a rate of 100 rpm to 900 rpm.

In another embodiment of the second method for producing a heterogeneous adjuvant formulation, the ratio of organic phase to aqueous phase of step (ii) ranges from 1:4 to 1:8. In a preferred aspect, the ratio is 1.8.

In another embodiment of the second method for producing a heterogeneous adjuvant formulation, the organic solvent is removed before compounding of step (vi).

In another embodiment of the second method for producing a heterogeneous adjuvant formulation, removing the organic phase of the intermediate liposome of step (iv) and step (v) is by Tangential Flow Filtration (TFF). In one aspect, the TFF is TFF ultrafiltration and diafiltration. In another aspect, the TFF comprises membranes having a molecular weight cut-off (MWCO) ranging from 100-500 kDa.

In another embodiment of the second method for producing a heterogeneous adjuvant formulation, the concentrating of step (vi) is by Ultrafiltration.

In another embodiment of the second method for producing a heterogeneous adjuvant formulation, a buffer is added to the compounding of step (vi).

In another embodiment of the second method for producing a heterogeneous adjuvant formulation, the compounding of step (vi) is at a mixing speed of 300 rpm for 1 hour at RT. In one aspect, following the compounding of step (vii) the intermediate liposome is stored at RT without stirring for 24 hours.

In another embodiment of the second method for producing a heterogeneous adjuvant formulation, the final adjuvant formulation has a size range of 300 nm to 1000 nm.

In another embodiment of the second method for producing a heterogeneous adjuvant formulation, the final adjuvant formulation has a polydispersity of 0.4 to 1.0.

In another embodiment of the second method for producing a heterogeneous adjuvant formulation, the injecting of step (ii) is by pipette, pump or syringe injection.

In an embodiment of the second method for producing a heterogeneous adjuvant formulation, said at least one phospholipid is a mixture of dimyristoyl phosphatidylcholine (DMPC) and dimyristoyl phosphatidylglycerol (DMPG).

In an embodiment, said second method for producing a heterogeneous adjuvant formulation comprises a liposome bilayer comprising (a) monophosphoryl lipid A (MPLA), (b) a saponin, and (c) a liposome composition comprising (i) at least one phospholipid selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG) and combination thereof, and (ii) cholesterol wherein the molar ratio of cholesterol to phospholipid is greater than 1,

comprises the steps of:
(i) dissolving the phospholipids, cholesterol and MPLA in an organic solvent to form a lipid formulation an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) concentrating the intermediate liposome of step (iii);
(v) removing the organic phase of the intermediate liposome of step (iv); and
(vi) compounding the intermediate liposome of step (vi) with a saponin,
   thereby producing the heterogeneous adjuvant formulation.

This invention provides a third method for producing a heterogeneous adjuvant formulation comprising a liposome bilayer comprising (a) monophosphoryl lipid A (MPLA), (b) a saponin, and (c) a liposome composition comprising (i) at least one phospholipid selected from at least one phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol),

said method comprising the steps of:
(i) preparing a lyopilized organic phase comprising the phospholipids, cholesterol and MPLA;
(ii) rehydrating the lyophilized organic phase of step (i) with an aqueous phase to form an intermediate liposome;
(iii) Downsizing of the intermediate liposome of step (ii) using microfluidizer;
(iv) compounding the intermediate liposome of step (iii) with a saponin,
   thereby producing the heterogeneous adjuvant formulation.

In one embodiment of the third method for producing a heterogeneous adjuvant formulation, the saponin is selected from the group consisting of QS-7, QS-18, QS-21, or a mixture thereof. In a preferred aspect, the saponin is QS-21.

In another embodiment of the third method for producing a heterogeneous adjuvant formulation, in step (i) the phospholipids, cholesterol and MPLA are dissolved in the organic solvent by sonication, heat, stirring or a combination thereof. In one aspect, the organic solvent comprises tert-butyl alcohol (TBA) or its mixture. In another aspect, the lipid organic phase is heated to a temperature from 25° C. to 65° C. In another aspect, the organic phase solution is lyophilized.

In another embodiment of the third method for producing a heterogeneous adjuvant formulation, the aqueous phase comprises water or a buffer. In one aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature from 20° C. to 70° C.

In another embodiment of the third method for producing a heterogeneous adjuvant formulation, the intermediate liposome of step (ii) is stirred at a rate of 100-1000 rpm.

In another embodiment of the third method for producing a heterogeneous adjuvant formulation, the size of the intermediate liposome in step (iii) is downsized with a microfluidizer and pressure at or about 18640 PSI.

In another embodiment of the third method for producing a heterogeneous adjuvant formulation, the organic solvent is removed before rehydration of step (ii). In one aspect, removing the organic solvent is by lyophilization.

In another embodiment of the third method for producing a heterogeneous adjuvant formulation, a buffer is added to the compounding of step (iv).

In another embodiment of the third method for producing a heterogeneous adjuvant formulation, the compounding of step (iv) is at a mixing speed of 300 rpm or by agitation for 1 hour at RT.

In another embodiment of the third method for producing a heterogeneous adjuvant formulation, the compounding of step (iv) the intermediate liposome is stored at RT without stirring for 24 hours.

In another embodiment of the third method for producing a heterogeneous adjuvant formulation, the final adjuvant formulation has a size>300 nm.

In another embodiment of the third method for producing a heterogeneous adjuvant formulation, the final adjuvant formulation has a polydispersity>0.4.

In an embodiment of the third method for producing a heterogeneous adjuvant formulation, said at least one phospholipid is a mixture of dimyristoyl phosphatidylcholine (DMPC) and dimyristoyl phosphatidylglycerol (DMPG).

In an embodiment, said third method for producing a heterogeneous adjuvant formulation comprises a liposome bilayer comprising (a) monophosphoryl lipid A (MPLA), (b) a saponin, and (c) a liposome composition comprising (i) at least one phospholipid selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG) and combination thereof, and (ii) cholesterol wherein the molar ratio of cholesterol to phospholipid is greater than 1,

comprises the steps of:
(i) preparing a lyopilized organic phase comprising the phospholipids, cholesterol and MPLA;
(ii) rehydrating the lyophilized organic phase of step (i) with an aqueous phase to form an intermediate liposome;
(iii) downsizing of the intermediate liposome of step (ii) using microfluidizer;
(iv) compounding the intermediate liposome of step (iii) with a saponin,
   thereby producing the heterogeneous adjuvant formulation.

This invention also provides an adjuvant formulation produced by any one of the methods described herein.

In one aspect, the liposome composition of the adjuvant formulation may comprise cholesterol at a mole percent concentration of over 50% (mol/mol), of about 55% to about 71% (mol/mol), or preferably about 55% (mol/mol). In one aspect, the liposome composition of the adjuvant formulation may comprise a phosphatidylcholine phospholipid (PC) selected from the group consisting of: dimyristoyl phosphatidylcholine (DMPC), dipalmitoyl phosphatidylcholine (DPPC), and distearyl phosphatidylcholine (DSPC). In another aspect, the liposome composition of the adjuvant formulation may comprise a phosphatidylglycerol phospholipid (PG) selected from the group consisting of: dimyristoyl phosphatidylglycerol (DMPG), dipalmitoyl phosphatidylglycerol (DPPG), and distearyl phosphatidylglycerol (DSPG). In a further aspect, the liposome composition of the adjuvant formulation may comprise a combination of (i) a phosphatidylcholine phospholipid (PC) selected from the group consisting of: dimyristoyl phosphatidylcholine (DMPC), dipalmitoyl phosphatidylcholine (DPPC), and distearyl phosphatidylcholine (DSPC), and (ii) a phosphatidylglycerol phospholipid (PG) selected from the group consisting of: dimyristoyl phosphatidylglycerol (DMPG), dipalmitoyl phosphatidylglycerol (DPPG), and distearyl phosphatidylglycerol (DSPG). The liposome composition of the adjuvant formulation may have a ratio of the PC to the PG (mol/mol) of about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. The liposome composition of the adjuvant formulation may comprise multi-lamellar vesicles (MLV) or small uni-lamellar vesicles (SUV), wherein small uni-lamellar vesicles are about 50 to about 100 nm in diameter, and wherein multi-lamellar vesicles are about 1 to about 4 μm in diameter. In a preferred embodiment, the liposome composition of the adjuvant formulation comprises a PC and a PG, wherein the PC is dimyristoyl phosphatidylcholine (DMPC) and the PG is dimyristoyl phosphatidylglycerol (DMPG), having a mole ratio of PC to PG (mol/mol) of about 9:1.

In another aspect, the liposome composition of the adjuvant formulation may comprise about 5 mg or less, about 4 mg or less, about 3 mg or less, about 2 mg or less, about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less of MPLA (total weight per ml liposome suspension). The liposome composition of the adjuvant formulation may have a MPLA:phospholipid mole ratio of about 1:5.6 to about 1:880, or about 1:88 to about 1:220. In a preferred embodiment, the liposome composition of the adjuvant formulation comprises a PC and a PG, wherein the PC is dimyristoyl phosphatidylcholine (DMPC) and the PG is dimyristoyl phosphatidylglycerol (DMPG), having a MPLA:phospholipid mole ratio of about 1:220, about 1:88 or about 1:5.6, preferably 1:88.

In a further aspect, the adjuvant formulation may have a content of saponin (total weight per ml liposome suspension) of about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less. In a preferred aspect, the adjuvant formulation comprises a content of saponin of about 0.2 to 0.4 mg/ml.

Also provided is an immunogenic composition comprising an immunogen and the adjuvant formulation. The immunogenic composition may typically comprise a physiologically acceptable vehicle. The immunogen of the immunogenic composition can be selected from the group consisting of a naturally-occurring or artificially-created protein, a recombinant protein, a glycoprotein, a peptide, a carbohydrate, a hapten, a whole virus, a bacterium, a protozoan, and a virus-like particle. A method of immunizing an animal comprising administering the immunogenic composition is also provided.

Further provided is a method of reducing toxicity of a saponin as an adjuvant or preparing an adjuvant formulation comprising adding a monophosphoryl lipid A (MPLA)-containing liposome composition to the saponin, wherein the liposome composition comprises i) a lipid bilayer comprising phospholipids in which the hydrocarbon chains have a melting temperature in water of ≥23° C. and ii) cholesterol at a mole percent concentration of greater than about 50% (mol/mol). The saponin may be selected from the group consisting of QS-7, QS-18, QS-21, and a mixture thereof. Preferably, the saponin is QS-21. The liposome composition may comprise cholesterol at a mole percent concentration of about 55% to about 71% (mol/mol), preferably about 55% (mol/mol).

### 1. Definitions

An “immunogen” is an agent capable of inducing humoral and/or cell-mediated immune response. The immunogen as described herein can be an antigen, a hapten, or an inactivated pathogen. An immunogenic composition as described herein can be, for example, a vaccine formulation.

As used herein, the term “homogeneous” shall mean a final adjuvant formulation comprising liposomes having a size range of about 30 nm-400 nm as determined by methods known in the art including, but not limited to, Dynamic light scattering (DLS), Transmission electron cryomicroscopy (e.g. cryo-TEM or cryo-EM), Nanoparticle Tracking Analysis (NTA, e.g. ViewSizer). A “homogeneous” adjuvant formulation may also mean a final adjuvant formulation comprising liposomes having a polydispersity index (PDI) of between about 0.05 to 0.5 or between about 0.05 to about 0.3, preferably about 0.3. Calculations used for the determination of size and PDI parameters may be found in the ISO standard documents 13321:1996 E and ISO 22412:2008 (Worldwide M.I. Dynamic Light Scattering, Common Terms Defined. Malvern Instruments Limited; Malvern, UK: 2011. Pp. 1-6. Inform White Paper). As used herein a “homogeneous” adjuvant formulation shall also mean a “monodisperse” adjuvant formulation.

As used herein, the term “heterogeneous” shall mean a final adjuvant formulation comprising liposomes having varying sizes ranging from about 30 nm to over 10 micrometers, about 30 nm to 4 micrometers, about 30 nm to 1400 nm, preferably about 30 nm to 1000 nm as determined by methods known in the art including, but not limited to, Dynamic light scattering (DLS), Transmission electron microscopy (e.g. cryo-TEM or cryo-EM), Nanoparticle Tracking Analysis (NTA, e.g. ViewSizer). A “heterogeneous” adjuvant formulation may also mean a final adjuvant formulation comprising liposomes having a polydispersity index (PDI)>0.5. Calculations used for the determination of size and PDI parameters may be found in the ISO standard documents 13321:1996 E and ISO 22412:2008 (Worldwide M.I. Dynamic Light Scattering, Common Terms Defined. Malvern Instruments Limited; Malvern, UK: 2011. pp. 1-6. Inform White Paper). As used herein a “heterogeneous” adjuvant formulation shall also mean a “polydisperse” adjuvant formulation.

“Liposomes” as used herein refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Liposomes, as they are ordinarily used, consist of smectic mesophases, and can consist of either phospholipid or nonphospholipid smectic mesophases. Smectic mesophase is most accurately described by Small, HANDBOOK OF LIPID RESEARCH, Vol. 4, Plenum, N.Y., 1986, pp. 49-50. According to Small, “[w]hen a given molecule is heated, instead of melting directly into an isotropic liquid, it may instead pass through intermediate states called mesophases or liquid crystals, characterized by residual order in some directions but by lack of order in others. In general, the molecules of liquid crystals are somewhat longer than they are wide and have a polar or aromatic part somewhere along the length of the molecule. The molecular shape and the polar-polar, or aromatic, interaction permit the molecules to align in partially ordered arrays. These structures characteristically occur in molecules that possess a polar group at one end. Liquid crystals with long-range order in the direction of the long axis of the molecule are called smectic, layered, or lamellar liquid crystals. In the smectic states the molecules may be in single or double layers, normal or tilted to the plane of the layer, and with frozen or melted aliphatic chains.”

Lipid A is a set of complex, heavily acylated and amidated diglucosamine diphosphate molecules and is the lipid moiety common to all lipopolysaccharides (LPS; also known as endotoxin) from Gram-negative bacteria. LPS covers virtually the entire outer surface of all Gram-negative bacteria, and lipid A anchors the LPS into the outer lipid surface of the bacterium. The O-polysaccharide portion of LPS in wild-type smooth bacteria is linked to a relatively conserved core oligosaccharide that is expressed in rough mutants, and this in turn is linked to lipid A through highly conserved 2-keto-3-deoxyoctanoic acid sugars that are unique chemical structures sometimes required for bacterial viability and found only in LPS. See, e.g., Alving et al., 2012, Expert Rev. Vaccines 11: 733-44. “Monophosphoryl lipid A” is a lipid A congener in which the glucosamine-1-phosphate group on the polar head group has been removed. Numerous congeners of MPLA also exist.

The “mole percent concentration of cholesterol” of a liposome composition as used herein refers to the ratio of Cholesterol:total phospholipid (i.e., phosphatidylcholine and phosphatidylglycerol) originally used in the preparation of the liposome composition.

A “physiologically acceptable vehicle” as used herein refers to a vehicle that is suitable for in vivo administration (e.g., oral, transdermal or parenteral administration) or in vitro use, i.e., cell culture. Exemplary physiologically acceptable vehicles can be those physiologically acceptable constituents of liposomes as disclosed in U.S. Pat. Nos. 4,186,183 and 4,302,459.

“Preferred” and “preferably” as used herein are to be construed for purposes of claim construction in Europe only. The terms should be read out of or omitted from the construction of the sentences and paragraphs in which they appear for purposes of U.S. claim construction.

The term “about” as used herein refers to ±5% of the referenced value.

Unless defined otherwise, all technical and scientific terms used herein have the same meaning as commonly understood by one of ordinary skill in the art. It must be noted that as used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

### 2. Saponin

For the present embodiments, a suitable saponin is Quil A, its derivatives thereof, or any purified component thereof (for example, QS-7, QS-18, QS-21, or a mixture thereof). Quil A is a saponin preparation isolated from the South American tree Quillaja saponaria Molina and was first found to have adjuvant activity. Dalsgaard et al., 1974, Archiv. für die gesanite Virusforschung, 44: 243-254. Purified fragments of Quil A have been isolated by HPLC (EP 0 362 278), including, for example, QS-7 and QS-21 (also known as QA7 and QA21, respectively). QS-21 is the 21st fraction purified from the sap of Quillaja saponaria tree. QS-21 has been shown to induce CD8+ cytotoxic T cells (CTLs), Th1 cells, and a predominant IgG2a antibody response.

### 3. Monophosphoryl Lipid A (MPLA)-Containing Liposomes (L(MPLA))

Liposomes are closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose, hydroxyethylcellulose, polyethyleneglycol, polyaspartamide and hydrophilic peptide sequences as described in U.S. Pat. Nos. 6,316,024; 6,126,966; 6,056,973; and 6,043,094. Liposomes can be made without hydrophilic polymers. Therefore, liposome formulations may or may not contain hydrophilic polymers.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phosphatidylglycerol, phosphatidylinositol, and sphingomyelin as disclosed in U.S. Pat. Nos. 6,056,973 and 5,874,104.

The vesicle-forming lipids may also be glycolipids, cerebrosides, or cationic lipids, such as 1,2-dioleyloxy-3-(trimethylamino)propane (DOTAP); N-[1-(2,3,-ditetradecyloxy)propyl]-N,N-dimethyl-N-hydroxyethylammonium bromide (DMRIE); N-[1(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 [N—(N′,N′-dimethylaminoethane) carbamoly]cholesterol (DCChol); or dimethyldioctadecylammonium (DDAB) also as disclosed in U.S. Pat. No. 6,056,973. Cholesterol may also be present in the proper range to impart stability to the liposome vesicle, as disclosed in U.S. Pat. Nos. 5,916,588 and 5,874,104. Additional liposomal technologies are described in U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479. These described liposomes and lipid-coated microbubbles, and methods for their manufacture. Thus, one skilled in the art, considering both the present disclosure and the disclosures of these other patents could produce a liposome for the purposes of the present embodiments. For the present embodiments, the liposome compositions typically contain about 1 mM to about 150 mM phospholipids.

Any of the above exemplary liposomes includes monophosphoryl lipid A (MPLA), or could be combined with other liposomes and lipid A (MPLA). MPLA alone can be toxic to humans and animals. However, when present in liposomes, the toxicity is not detected. See, e.g., Alving et al., 2012. MPLA serves as a potent adjuvant and serves to raise the immunogenicity of the liposome and peptides, proteins, or haptens associated with the liposome.

For the present embodiments, a monophosphoryl lipid A (MPLA)-containing liposome (L(MPLA)) may be one originally referred to as Walter Reed liposomes but now known as Army Liposome Formulation (ALF), as a vaccine adjuvant. See, e.g., Alving et al., 2012. The ALF adjuvant liposomes comprise (1) a lipid bilayer comprising phospholipids in which the hydrocarbon chains have a melting temperature in water of ≥23° C., usually dimyristoyl phosphatidylcholine (DMPC, e.g. 1,2-dimyristoyl-sn-glycero-3-phosphocholine) and dimyristoyl phosphatidylglycerol (DMPG, e.g. 1,2-dimyristoyl-sn-glycero-3-phospho-(1′-rac-glycerol)); (2) cholesterol (Chol) as a stabilizer; and (3) monophosphoryl lipid A (MPLA) as an immunostimulator. In human clinical trials, the ALF-type liposomal adjuvant proved to be safe and potent in candidate vaccines to malaria, HIV-1, and cancer. See, e.g., Fries et al., 1992, Proc. Natl. Acad. Sci. USA, 89: 358-62; Alving, 2002, Vaccine, 20: S56-64. The particular composition of ALF to which QS-21 is added to form ALFQ comprises cholesterol at a mole percent concentration of greater than about 50% (mol/mol), preferably about 55% to about 71% (mol/mol), or more preferably about 55% (mol/mol). Additionally in the present embodiments, monophosphoryl lipid A (MPLA)-containing liposome (L(MPLA)) comprising a saponin (e.g. QS-21) may be a homogeneous LiNA-2 adjuvant formulation or a heterogeneous LiNA-2 adjuvant formulation, as described herein.

For the present embodiments, an L(MPLA) may comprise a phosphatidylcholine (PC) selected from the group consisting of dimyristoyl phosphatidylcholine (DMPC), dipalmitoyl phosphatidylcholine (DPPC), and disteaiyl phosphatidylcholine (DSPC). The L(MPLA) may also comprise a phosphatidylglycerol (PG) selected from dimyristoyl phosphatidylglycerol (DMPG), dipalmitoyl phosphatidylglycerol (DPPG), and distearyl phosphatidylglycerol (DSPG). The PC to PG ratio (mol/mol) of the liposome may be about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. The liposome may have a content of MPLA (total weight per ml liposome suspension) of about 5 mg or less, about 4 mg or less, about 3 mg or less, about 2 mg or less, about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less. Alternatively, the liposome may have a MPLA:phospholipid mole ratio of about 1:5.6 to about 1:880, preferably about 1:88 to about 1:220. Prior to the addition of a saponin, the liposome may comprise multilamellar vesicles (MLV) or small uni-lamellar vesicles (SUV). The small unilamellar vesicles may be about 50 to about 100 nm in diameter, while the multilamellar vesicles may be about 1 to about 4 μm in diameter.

### 4. Adjuvant Formulations Comprising MPLA-Containing Liposomes (L(MPLA) and Saponin

An adjuvant formulation known as AS01 (also known as AS01B or AS01E) was previously introduced by GlaxoSmithKline. In AS01, the lipid bilayer was comprised of a neutral lipid that is “non-crystalline” at room temperature, such as dioleoyl phosphatidylcholine, cholesterol, MPLA, and QS-21. See U.S. Pat. No. 10,039,823. During manufacture of AS01 small unilamellar liposomal vesicles (SUV) are first created and purified QS-21 is then added to the SUV. The QS-21 imparts unique properties in that it binds to the liposomal cholesterol where it causes perforations (holes) or other permanent structural changes in the liposomes. See, e.g., Paepenmuller et al., 2014, Int. J. Pharm., 475: 138-46. A reduced amount of free QS-21 presumably resulted in reduced local injection pain often caused by free QS-21. See, e.g., Waite et al., 2001, Vaccine, 19: 3957-67; Mbawuike et al., 2007, Vaccine, 25: 3263-69. The AS01B formulation was created for vaccines where the induction of a yet stronger T-cell-mediated immune response is required.” See Garçon et al., 2007, Expert. Rev. Vaccines, 6: 723-39. The AS01 formulation is being developed as an adjuvant for a variety of vaccines. See Garcon & Mechelen, 2011, Expert. Rev. Vaccines, 10: 471-86. The AS01 formulation, as described in U.S. Pat. No. 10,039,823, may contain cholesterol (sterol) at a mole percent concentration of 1-50% (mol/mol), preferably 20-25% (mol/mol).

The present invention provides an adjuvant formulation produced by the methods described herein comprising a monophosphoryl lipid A (MPLA)-containing liposome composition and at least one saponin, wherein the liposome composition comprises i) a lipid bilayer comprising phospholipids (e.g., dimyristoyl phosphatidylcholine (DMPC) and/or dimyristoyl phosphatidylglycerol (DMPG)) in which the hydrocarbon chains have a melting temperature in water of ≥23° C., and ii) cholesterol at a mole percent concentration of greater than about 50% (mol/mol), or preferably about 55% to about 71% (mol/mol), or more preferably about 55% (mol/mol). At least these two features are distinct from those of AS01 as discussed above. The saponin may be selected from QS-7, QS-18, QS-21, or a mixture thereof, or the saponin preferably may be QS-21. The adjuvant formulation may contain about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less of saponin per ml liposome suspension. In a preferred aspect, the adjuvant formulation comprises a content of saponin of about 0.2 to 0.4 mg/ml.

In one aspect, said adjuvant formulation comprises a liposome composition comprising i) a lipid bilayer comprising phospholipids and ii) cholesterol, where the molar ratio of cholesterol to phospholipids is greater than about 1.

In a preferred aspect, the liposome composition of the adjuvant formulation comprises a PC and a PG, wherein the PC is dimyristoyl phosphatidylcholine (DMPC) and the PG is dimyristoyl phosphatidylglycerol (DMPG), having a mole ratio of PC to PG (mol/mol) of about 9:1.

In a further aspect, the liposome composition of the adjuvant formulation may have a MPLA:phospholipid mole ratio of about 1:5.6 to about 1:880, or about 1:88 to about 1:220. In a preferred embodiment, the liposome composition of the adjuvant formulation comprises a PC and a PG, wherein the PC is dimyristoyl phosphatidylcholine (DMPC) and the PG is dimyristoyl phosphatidylglycerol (DMPG), having a MPLA:phospholipid mole ratio of about 1:220, about 1:88 or about 1:5.6, preferably 1:88.

In another embodiment, the invention provides an adjuvant formulation produced by the methods described herein wherein the adjuvant formulation comprises unilamellar liposomes having a liposome bilayer that consists of: (a) at least one phosphatidylcholine (PC) and/or phosphatidylglycerol (PG), as phospholipids, selected from the group consisting of: dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG); (b) cholesterol; (c) monophosphoryl lipid A (MPLA); and (d) a saponin; and wherein the mole ratio of the cholesterol (b) to the phospholipids (a) is greater than about 50:50; and wherein the unilamellar liposomes have a median diameter size in micrometer range as detected by light scattering analysis. In one aspect, the saponin is QS-7, QS-18, QS-21, or a mixture thereof, preferably QS-21. In another aspect, the mole ratio of the cholesterol (b) to the phospholipids (a) is about 55:45 to about 71:29. In another aspect, the mole ratio of the cholesterol (b) to the phospholipids (a) is about 55:45. In another aspect, dimyristoyl phosphatidylcholine (DMPC) is selected as a phospholipid, wherein additionally dimyristoyl phosphatidylglycerol (DMPG) is selected as a phospholipid. In another aspect, both a PC and a PG are selected as phospholipids, and wherein the ratio of the PC to the PG (mol/mol) is about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. In another aspect, the invention provides a liposome suspension comprising the adjuvant formulation described herein and phosphate-buffered saline (PBS), pH 7.4, wherein the liposome suspension comprises (i) 1.272 mM to 50 mM of the phospholipids (a), and (ii) about 5 mg/ml or less of the MPLA (c). In another aspect, the mole ratio of the MPLA (c) to the phospholipids (a) is about 1:5.6 to about 1:880. In a further aspect, the invention provides a liposome suspension comprising the adjuvant formulation described herein and phosphate-buffered saline (PBS), pH 7.4, wherein the liposome suspension comprises (i) 1.272 mM to 50 mM of the phospholipids (a), and (ii) about 1 mg/ml or less of the saponin (d). In another aspect, the mole ratio of the MPLA (c) to the phospholipids (a) is about 1:88 to about 1:220.

Examples of adjuvant formulations comprising an MPLA-containing liposome composition and at least one saponin (e.g. QS-21) made by the methods described herein include, but are not limited to, adjuvant formulations described in U.S. Pat. No. 10,434,167 (e.g. ALFQ) and two Liposomal Novel Adjuvant-2 (LiNA-2) adjuvant formulations described herein, namely a LiNA-2 homogeneous adjuvant formulation and a LiNA-2 heterogeneous adjuvant formulation. In one aspect the LiNA-2 adjuvant comprises a synthetic TLR4 agonist, monophosphoryl lipid A (MPLA), a triterpenoid glycoside saponin (QS-21), 1,2-dimyristoyl-sn-glycero-3-phosphocholine also known as dimyristoyl phosphatidylcholine (DMPC), 1,2-dimyristoyl-sn-glycero-3-phospho-(1′-rac-glycerol) also known as dimyristoyl phosphatidylglycerol (DMPG), and cholesterol. In a preferred aspect, LiNA-2 comprises MPLA (e.g. 3D-PHAD®), QS-21, DMPC, DMPG and cholesterol. In another preferred aspect, LiNA-2 comprises MPLA (e.g. 3D-PHAD®), QS-21, DMPC, DMPG and cholesterol in a phosphate buffer comprising sodium chloride (NaCl). In one aspect, the LiNA-2 adjuvant is designed to reconstitute lyophilized powder formulation for administration. In another aspect, the LiNA-2 is designed to be mixed with a liquid formulation for administration.

In a further aspect, LiNA-2 homogeneous or LiNA-2 heterogeneous adjuvant formulations may be LiNA-2 at 1× concentration (1XLiNA-2) or LiNA-2 at 2× concentration (2XLiNA-2) as compared to ALFQ concentration as described herein. In one embodiment, the adjuvant formulation is ALFQ comprising (i) 7.0 mg/mL DMPC, (ii) 0.78 mg/ml DMPG, (iii) 5.4 mg/ml cholesterol, (iv) 0.2 mg/mL MPLA (3D-PHAD), and (v) 0.1 mg/ml QS-21. In another embodiment, the adjuvant formulation is 1XLiNA-2, wherein the 1XLiNA-2 may be homogeneous or heterogeneous, comprising (i) 14±7 mg/mL DMPC, (ii) 1.6±0.8 mg/ml DMPG, (iii) 11±6 mg/ml cholesterol, (iv) 0.40±0.20 mg/mL MPLA (3D-PHAD), and (v) 0.20±0.10 mg/ml QS-21. In a further embodiment, the adjuvant formulation is 2XLiNA-2, wherein the 2XLiNA-2 may be homogeneous or heterogeneous, comprising (i) 28±14 mg/mL DMPC, (ii) 3.2±1.6 mg/ml DMPG, (iii) 22±11 mg/ml cholesterol, (iv) 0.80±0.40 mg/mL MPLA (3D-PHAD), and (v) 0.40±0.20 mg/ml QS-21.

### 5. Uses of Adjuvant Formulations Comprising MPLA-Containing Liposomes (L(MPLA) and Saponin

The adjuvant formulations of the present embodiments may be used to mix with an immunogen to obtain an immunogenic composition, for example, a vaccine. The immunogenic composition may comprise a physiologically acceptable vehicle, for example, any one of those described in U.S. Pat. No. 5,888,519. The immunogenic composition may comprise naturally-occurring or artificially-created proteins, recombinant proteins, glycoproteins, peptides, carbohydrates, nucleic acids, haptens, whole viruses, bacteria, protozoa, or virus-like particles, or conjugates thereof as the immunogen. The immunogenic composition may be suitably used as a vaccine for chickenpox or shingles, human respiratory syncytial virus infection (RSV), Cytomegalovirus infection (CMV), Human metapneumovirus, Human parainfluenza viruses type 1 or type 3, Lyme disease, Streptococcus pneumonia, Clostridioides difficile, Escherichia coli or Klebsiella pneumoniae, influenza, HIV-1, Hepatitis A, Hepatitis B, Human Papilloma virus, Meningococcal type A meningitis, Meningococcal type B meningitis, Meningococcal type C meningitis, Tetanus, Diphtheria, Pertussis, Polio, Haemophilus influenza type B, Dengue, Hand Foot and Mouth Disease, Typhoid, Pneumococcus, Japanese encephalitis virus, Anthrax, Shingles, Malaria, Norovirus, or cancer. The immunogenic composition may be suitably used in methods for treating or preventing a disease or infection in a subject, preferably wherein the subject is a human, caused by a pathogen associated with an infectious disease wherein the pathogen is selected from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 0157:H7, 0111 and 0104:H4, Escherichia coli Fimbrial antigen H, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Klebsiella pneumoniae, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella-zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

The present invention provides an immunogenic composition comprising an immunogen and an adjuvant formulation as described herein.

### EXAMPLES

In order that this invention may be better understood, the following examples are set forth. These examples are for purposes of illustration only and are not to be construed as limiting the scope of the invention in any manner. The following Examples illustrate some embodiments of the invention.

In the following Examples, the dimyristoyl phosphatidylcholine (DMPC), dimyristoyl phosphatidylglycerol (DMPG), and synthetic monophosphoryl lipid A (MPLA) (3D-PHAD™) may be purchased from Avanti Polar Lipids (Alabaster, Ala., USA). Purified QS-21 may be purchased from Desert King International (San Diego, Calif., USA).

Also, in the following Examples, cholesterol content may be analyzed to confirm the cholesterol, and indirectly, the phospholipid concentration of the liposome composition by the established methods. See, e.g., Zlatkis et al., 1953, J. Lab. Clin. Med., 41: 486-492. The cholesterol concentration of the liposome composition may be determined from a cholesterol standard curve.

### Example 1: Homogeneous Adjuvant Formulation Preparation

Liposomes of varying sizes were generated using a solvent injection process. The liposomes were formed by dissolving lipids (DMPG, DMPC, cholesterol and MPLA) in organic solvent (ethanol or isopropyl alcohol) by sonication, heat or combination of both (organic phase). The lipids in organic solvent (organic phase) were heated up to 65° C. The organic phase was pumped into a buffer (aqueous phase) kept either at RT (22° C.-25° C.) or in a heated state (up to 60° C.). The buffer (aqueous phase) is 10 mM phosphate at pH 6.2 containing 150 mM NaCl. The introduction of lipids (organic phase) can be as slow as 0.5 mL/min to 400 mL/mim or a quick addition of lipids (organic phase) to buffer (aqueous phase).

The size of the liposome formed had a size ranging from 40 nm to 400 nm by controlling the rate of addition of lipid to buffer along with altering the ratio of organic solvent to aqueous phase (buffer or water). The ratio tested ranged from 1:4 to 1:16. Liposome formation was controlled by stirring the buffer at 700 rpm along with controlling the temperature of the buffer, either at room temperature or in a heated state. The liposomes formed during this process were reduced in size using high pressure extruders or microfluidic homogenizers. The size was controlled by selection of mesh sizes ranging from 50 nm to 120 nm, or use of homogenization pressure between 17000-24000 PSI. After this downsizing process, the intermediate liposomes formed ranged from 40-100 nm, enabling sterile filtering.

Table 1 below provides data from the multiple batches of intermediate liposomes formed.

TABLE 1

| Intermediate Liposome batches | Size (nm) | PDI |
|---|---|---|
| Batch-1 | 93.58 | 0.09 |
| Batch-2 | 88.83 | 0.069 |
| Batch-3 | 94.4 | 0.06 |
| Batch-4 | 90.65 | 0.094 |
| Batch-5 | 95.08 | 0.074 |
| Batch-6 | 91.93 | 0.057 |
| Batch-7 | 96.07 | 0.125 |
| Batch-8 | 56.89 | 0.101 |
| Batch-9 | 93.01 | 0.078 |
| Batch-10 | 68.77 | 0.087 |

The organic solvent in the liposomes may be removed prior to downsizing or after downsizing. Tangential flow filtration is used to remove the ethanol using hollow fiber membranes or cassette membranes. The concentration of the final product may be adjusted by controlling the ultrafiltration and diafiltration steps. The molecular weight cut-off (MWCO) of the membranes used for TFF range from 100-500 kDa. Following TFF, the liposomes are resuspended in buffer and passed through a bioburden reduction filter (0.45 micron) and a sterile filter (0.22 micron).

The intermediate liposome containing MPLA is compounded (the process of combining, mixing, or altering) in an aseptic (sterile environment) manner with a saponin (e.g QS-21) to generate the final adjuvant drug product. The size of the final adjuvant has a range of 50-200 nm with a polydispersity of 0.05 to 0.3. During compounding the mixing speed was set at 300 rpm. The process of mixing ranged from 1 hr to 24 hrs, wherein the mixing could be done at room temperature or at 2-8° C.

### Example Preparation of Organic Phase:

Lipids (DMPC, DMPG and cholesterol) at twice the final concentration (1×) quantities were weighed and added to glass vials and quantum satis (qs) with required volume of Ethanol. 45 ml 2× lipid solution was added to 360 ml of 10 mM phosphate buffer with 150 mM NaCl at pH 6.2 in 1:8 ratio. Heated a 50 ml glass syringe at 50° C. The mass of the lipids was chosen according to Table 2 for preparation of a 50 ml of lipid stock solution: 40 mg MPLA (3D-PHAD) added to 2 ml ethanol into each vial and sonicated while heating in a bath sonicator at 48° C. for 30 min. Afterward, these MPLA (3D-PHAD) solutions were added to the beaker of the other lipids weighted and the total volume of the lipid stock solution was adjusted to 50 ml.

TABLE 2
Lipid Stock (in 50 mL Ethanol)

| Chemical | Concentration | Mass needed | Measured Mass |
|---|---|---|---|
| Name | (mg/ml)-2X lipid sln | (mg) | (mg) |
| DMPC | 28 | 1400 | 1403.1 |
| DMPG | 3.16 | 158 | 158.2 |
| Cholesterol | 21.64 | 1082 | 1083.2 |
| MPLA | 0.8 | 40 | 40 (5 mg * |
| | | | 8 vials) |

### Steps for Preparation of a Batch

1. Pour 360 ml of PB into a 500 mL Pyrex media storage bottle containing a white magnet.
2. Set the syringe pump on (Rate=900 μl/min, Syringe diameter=X mm (pick the proper diameter of the syringe based on the pump manual, Volume=45.00 ml).
3. Wrap the syringe with the flat to spiral heating pad and connect the pad to the temperature controller/the heater. Set the heater temperature at 50° C.
4. Take out the 50 mL ethanol lipid solution into the 50 ml wrapped glass syringe using a needle and remove any bubble or gas inside. Remove the needle and cap the syringe immediately. Cut 25 cm tubing-size 16 and cap it from one side.
5. Remove the cap from the syringe and immediately connect the opened end of tubing tip to the syringe.
6. Place the PB containing media storage bottle on the hotplate while having the magnet stirring inside at −900 rpm at RT (22° C.-25° C.).
7. Open the other ending of the tube and put it in the media storage bottle.
8. Fill the tubing with the lipid solution. Avoid any bubble forming inside and cap the tubing ending again once making sure the lipid is ready to be added to the PB buffer.
9. Open the tubing ending and place it in the media storage bottle. Start injection at 900 ul/min till 45.00 ml of the lipid solution is added to the reaction bottle.
10. Measure the size by DLS. 950 μl of milli Q-Water and 50 μl of the sample. (DF=20).

The resulting sample was extrused using a 100 ml extruder. First cycle one 100 nm membrane was used 2^{nd} and 3^{rd} cycles were done using a 50 nm membrane for each cycle.

The pressure used for extrusion was between 400 to 500 psi for 2^{nd} and 3^{d} cycle and between 600 to 700 psi for first cycle of the extrusion.

TFF was done on the extruded sample using a 100, 300 or 500 kDa MWCO mPES hollow fiber membrane. 275 ml sample was concentrated back to 50 ml and 10 DV wash was done on it (500 ml buffer used) after which the sample was concentrated back to 35 mL.

The extruded liposomes were loaded on to a hollow fiber tangential flow system (500 kDa mPES membrane). Concentrate the total volume loaded to a final volume to match that of lipid solution. Dia-volume washes (10-12) were done to remove all of the ethanol. Final size and concentration measurements were done before sterile filtering the intermediate liposomes. Intermediate liposomes should be at twice the concentration of the final product. Intermediate liposomes were stored at 2-8° C. before sterile filtering

The appropriate amount of QS-21 (0.5 mg/mL-2 mg/mL) was weighed and reconstituted using 10 mM phosphate buffer containing 150 mM sodium chloride at pH 6.2 to mix with liposome intermediate formed and achieve a final target concentration of either 0.2 mg/mL or 0.4 mg/mL QS-21 in the adjuvant drug product. The solution of QS-21 was sterile filtered and compounded with the intermediate liposome to generate the final adjuvant drug product.

Table 3 summarizes the size and PDI of multiple batches of the final adjuvant formulation produced according to the process described in this Example.

TABLE 3

| Final Adjuvant formulation | Size (nm) | PDI |
|---|---|---|
| Batch-1 | 102.6 | 0.204 |
| Batch-2 | 135.3 | 0.464 |
| Batch-3 | 110.8 | 0.276 |
| Batch-4 | 111.2 | 0.279 |
| Batch-5 | 100.1 | 0.141 |
| Batch-6 | 112.1 | 0.218 |
| Batch-7 | 103.6 | 0.169 |
| Batch-8 | 165 | 0.165 |
| Batch-9 | 114.4 | 0.281 |
| Batch-10 | 105.9 | 0.223 |

### Example 2: Homogeneous Adjuvant Formulation Preparation: A Process Using Solvent Injection to Prepare 40-150 nm Intermediate Liposome without Downsizing

### A. Preparation of the Intermediate Liposome 40-150 nm

Weighed the lipids (DMPG, DMPC, cholesterol and MPLA) in a fresh glass bottle and added 100 ml ethanol to make lipid stock (organic phase) as described in Table 4. The lipid stock was stirred and the glass bottle was heated at 45° C. in a water bath in a beaker. After the lipids were dissolved, the lipid stock was diluted by 2.5 fold using ethanol.

TABLE 4
Lipid Stock (in 100 mL Ethanol)

| Component | MPLA - 3D-PHAD | DMPC | DMPG | Chol |
|---|---|---|---|---|
| Actual weights | 80 | 2800.7 | 316.3 | 2164.1 |
| added (mg) | | | | |

The size of the resulting liposomes was screened using NanoAssemblr having the conditions listed in Table 5. The flow ratio 4:7 was selected for further development of the syringe injection process described in section B below.

TABLE 5

| Temperature | Flow | Total | Intermediate | |
|---|---|---|---|---|
| aq/org | rate ratio | flow rate | liposome | |
| (° C.) | org/aq | (ml/min) | Size (nm) | PDI |
| 45/45 | 1:8 | 12 | 40 | 0.162 |
| 45/45 | 1:8 | 12 | 39 | 0.144 |
| 45/45 | 1:4 | 12 | 47 | 0.124 |
| 45/45 | 1:2 | 12 | 62 | 0.023 |
| 45/45 | 4:7 | 12 | 87 | 0.070 |
| 45/45 | 2:3 | 12 | 107 | 0.084 |
| 45/45 | 2:3 | 17 | 111 | 0.054 |
| 45/45 | 4:5 | 12 | 134 | 0.055 |
| 45/45 | 1:1 | 12 | 127 | 0.019 |
| 55/55 | 5:1 | 12 | 47 | 0.06 |

B. Syringe Injection [Using Intermediate Liposome Having 40-150 nm]

The lipid solution in ethanol described in section A above (organic phase) was heated to 40-65° C. in a beaker placed on a hot plate and loaded in a 60 mL plastic/glass syringe (with 10 mL extra space). The syringe was wrapped in a heating pad to maintain the temperature between 45-55° C. The total 60 mL of the lipid solution was loaded in the organic syringe. The water was heated to 45° C. in a beaker placed on a hot plate and loaded into a 120 mL plastic syringe. The syringe was maintained at 45° C. using a heating pad The aqueous phase and organic phase are connected by a Upchurch Stainless Steel Tee assembly (Idex). Both syringes were attached to pumps which were started simultaneously using the software to generate 20 ml of liposome following the conditions in Table 6. The resulting liposome was 40-140 nm with a PDI of 0.04-0.2.

The resulting liposomes were purified by TFF and concentrated to prepare a liposome intermediate with a size of 40-91 nm and a PDI of 0.05-0.1.

TABLE 6

| | Volume of | Volume of |
|---|---|---|
| Total volume: | aqueous phase: | organic phase: |
| 150 ml | 95.45 ml | 54.55 ml |
| | Flow rate of | Flow rate of |
| Total flow rate: | aqueous phase: | organic phase: |
| 12 ml/min | 7.636 ml/min | 4.363 ml/min |

### C. Preparation of QS-21

11 mg of QS-21 was weighed in a 50 mL conical tube. 6.629 mL of PBS buffer (10 mM phosphate, 150 mM NaCl, pH 6.2) was added to make 1.5 mg/mL QS-21 solution. The QS-21 solution was shaken until the QS-21 dissolved completely. QS-21 stock solution was filtered using a 0.22 uM PES steri-flip filter. The stock solution was collected in the 50 mL conical tube and stored at 5 C covered in aluminum foil.

### D. Preparation of Adjuvant Product

1 mL of the liposome intermediate from process B was mixed with 0.219 mL of QS-21 solution and 0.421 ml PBS buffer. The combined solution was immediately covered with aluminum foil to avoid light exposure. It was then shaken for 1 hour at RT at 350 rpm then stored for 23 hr at RT without agitation. The final formulation with a size of 102 nm and a PDI of 0.18 was saved at 2-8° C. as the final formulation. The concentration of final adjuvant drug product contained a target range of 0.2-0.4 mg/mL QS-21.

The defined process can be scaled up using a pump based set up where flow rates and mixing parameters are controlled as done in the syringe set up set forth in section B of this Example.

### Example 3: Methods for Heterogeneous Adjuvant Formulation Preparation

### A. Method I: A Process Using Solvent Injection and Microfluidizer

### 1) Intermediate Preparation

Weighed the lipids in a fresh glass bottle and added 100 ml lipid stock in ethanol. The amount of lipids as per the table mentioned below. A magnetic stir bar was placed in the bottle. The glass bottle was heated at 65° C. in a water bath in a beaker. After the lipids were dissolved, the sample was diluted by 2.5 fold using ethanol.

TABLE 7

| | Component | | | | |
|---|---|---|---|---|---|
| | MPLA - | | | | Ethanol |
| | 3D-PHAD | DMPC | DMPG | Chol | (ml) |
| Actual weights | 80 | 2800.7 | 316.3 | 2164.1 | 100 |
| added (mg) | | | | | |

The lipid solution was heated up to 65° C. in a beaker placed on a hot plate and loaded in a 60 mL plastic syringe (with 10 mL extra space). The syringe was maintained at 65° C. with the help of heating pad wrapped around it which was secured with the help of zipties. 60 mL of the lipid solution was loaded in the organic syringe. The WFI was heated upto 60° C. in a beaker placed on a hot plate and loaded in two 100 mL plastic syringe (with 10 mL extra space). The syringe was maintained at 60° C. with the help of heating pad wrapped around it which was secured with the help of zipties.

The settings used on the Adagio software/system for organic phase are set forth in Table 5.

TABLE 8

| Settings | Organic solution | WFI value assigned |
|---|---|---|
| | value assigned | |
| Syringe model | Becton Dickinson | Becton Dickinson Glass |
| | Plasti-Pak 60 mL | (all types) 100 mL |
| No. of syringes | 1 | 1 |
| Flow directions | Withdraw | Infuse |
| Flow rate | 1.330 mL/min | 10.668 mL/min |
| Volume | 22.5 mL | 90 mL |
| Time | 00:16:52 | 00:16:52 |

The lipid solution in ethanol described in section A above (organic phase) was heated to 65° C. in a beaker placed on a hot plate and loaded in a 65 mL plastic/glass syringe. The syringe was wrapped in a heating pad to maintain the temperature˜65° C. The total 60 mL of the lipid solution was loaded in the organic syringe. The water was heated to 60° C. in a beaker placed on a hot plate and loaded into a 120 mL plastic syringe. The syringe was maintained at 60° C. using a heating pad. The aqueous phase and organic phase are connected by a Steel upchurch Tee-assembly. Both syringes were attached to pumps which were started simultaneously using the software to generate 20 ml of liposome following the conditions in Table 8. Initial 5% of the run samples are discarded. Ensure that the sample collected is not turbid and collect in a glass container. The resulting liposome was 40-70 nm with a PDI of 0.04-0.2.

1200 ml sample with a size˜45 nm and a PDI ˜0.04-0.1 combined from a few different batches was loaded gradually to a TFF system at the concentration step to obtain 60 mL liposome. During diafiltration step, the flowrate from 500 ml/min reduced to 400, 300, 200 and 100 at 10× point, then kept stable 10× to 20× at 100 ml/min, feed 14 psi, TMP 7.5 psi. About 10 times the dilution volume of PBS buffer, pH 6.2 was used for buffer exchange starting at 100 ml/min, gradually increased flowrate at 200, 300, 400, 500 ml/min, then kept 500 ml min, feed 12 psi, TMP 8 psi. Harvested about 200 ml/min flow rate for the retentate only to get 60-64 mL. The collected samples after TFF were filtered using steriflip 50 mL filtration system with 220 nm membrane to prepare liposome with a size of ˜55 nm and a PDI of ˜0.13.

The liposomes solution further passed through a Y shaped chamber of the microfluidizer for about 10 passes followed by filtration to prepare the intermediate with a size of 58 nm and PDI of 0.12.

### 2) Preparation of QS-21

11 mg of QS-21 was weighed in a 50 mL conical tube. 6.629 mL of PBS buffer (10 mM phosphate, 150 mM NaCl, pH 6.2) was added to make 1.5 mg/mL QS-21 solution. The QS-21 solution was shaken until the QS-21 dissolved completely. QS-21 stock solution was filtered using a 0.22 uM PES steri-flip filter. The stock solution collected in the 50 mL conical tube and stored in 5° C. covered in aluminum foil

### 3) Preparation of Adjuvant Product

Mixed 1 mL the intermediate and 0.219 mL of QS-21 solution with 0.421 ml PBS buffer. The combined solution was immediately covered with aluminum foil to avoid light exposure. It was then shaken for 1 hour at RT at 350 rpm then stored for 48 hr at RT without agitation. The final formulation with a size of 645 nm and a PDI of 0.62 was saved at 2-8° C. as final formulation. The defined process scaled up using a pump based set up where flow rates and mixing parameters are controlled as done in the syringe set up.

### B. Method II: Ethyl Acetate Injection Process

### 1) Intermediate Preparation

Lipids were weighed and added to glass vials with required volume of 155 mL ethyl acetate and 5 mL isopropanol (IPA). The lipids are incubated in water bath at 55° C. for 30 mins to make a clear solution. The clear lipid solution is loaded into 50 mL glass syringe wrapped with a heating pad to maintain the temperature at 55° C. controlled by temperature sensor. 4.5 mL lipid solution was added into 36 mL of PBS pH 6.2 buffer using a syringe pump with a flow rate of 20 mL/min. The formulation was purified and concentrated by TEE to 5-6 mL followed by 10 DV of PBS pH 6.2. It was further concentrated by centrifugation to 1.7 mL to prepare the liposome intermediate.

TABLE 9
The mass of the lipids was chosen according to the table
below for preparation of 160 ml of lipid solution

| Name | Actual amount (mg) |
|---|---|
| DMPC | 1127.2 |
| DMPG | 129.0 |
| Cholesterol | 869.3 |
| MPLA | 34.1 |

TABLE 10
Size and distribution of different processes

| | | | | | | | | Size | |
|---|---|---|---|---|---|---|---|---|---|
| | | | | | | Size | | with | PDI |
| | | EA and | | | | after | PDI | QS- | with |
| | Addition | Ethanol of | | Size, | | TFF/Con, | after | 21, | QS- |
| Sample | method | lipid solution | Organic/Buffer | nm | PDI | nm | TFF/Con | nm | 21 |
| 1 | Pipette | EA only, turbid | 4 ml/0.5 ml | 756.9 | 0.847 | | | | |
| | | at 50-55 C., | | | | | | | |
| | | 0.5X Lipids | | | | | | | |
| 2 | Pipette | 3% IPA in EA, | 4 ml/0.5 ml | 622.7 | 0.627 | | | | |
| 3 | Pipette | Clear at 50- | 4 ml/0.5 ml | 877.1 | 0.641 | | | | |
| 4 | Pipette | 55 C., 0.5X | 4 ml/0.5 ml | 580.1 | 0.726 | | | | |
| 5 | Syringe, 20 | Lipids | 36 ml/4.5 ml | 805.9 | 0.774 | 549 | 0.650 | 564 | 0.957 |
| | ml/min | | | | | | | | |
| 6 | Syringe, 20 | | 36 ml/4.5 ml | 637.1 | 0.604 | 462 | 0.695 | 582 | 0.623 |
| | ml/min | | | | | | | | |
| 7 | Syringe, 20 | | 36 ml/4.5 ml | 675.7 | 0.649 | 757 | 0.993 | 844 | 0.584 |
| | ml/min | | | | | | | | |

### 2) Preparation of Adjuvant Product

0.2 mL of 1.2 mg/mL QS-21 solution was added into 0.4 mL of the intermediate followed by 2.25 mL PBS Buffer pH 6.2. Shake the bottle under 300 rpm, room temperature (RT) for 1 hour by a shaking bed. Incubate the solution under RT for additional 24 hours. The final adjuvant formulation was saved at 5° C.

TABLE 11
Size and PDI after each process

| Process Stage | Size (nm) | PDI |
|---|---|---|
| Ethyl acetate injection | 637 | 0.60 |
| TFF | 478 | 0.552 |
| Concentration by centrifugation | 462 | 0.695 |
| QS-21 addition | 582 | 0.623 |
| 1 week at 5° C. | 542 | 0.649 |
| 2 weeks at 5° C. | 567 | 0.930 |

### C. Method III: A Process Using Rehydration of Freeze-Dried Lipids and Microfluidizer

### 1) Intermediate Preparation

Manufacture of the formulation involves weighing lipids MPLA (3D-PHAD), DMPC, DMPG, and cholesterol and dissolving down to with pre-warmed (55-65° C.) neat tert-butyl alcohol (TBA) and stirring at 300 rpm for >30 mins for complete dissolution. Final solution is loaded onto the lyophilizer for freeze/dry process. Following complete freezing step, the lyophilized lipids are then resuspended with ⅓ volume of 55-60° C. pre-warmed buffer (PBS, pH 6.2) for rehydration. The concentration can be controlled by varying the buffer volume. The rehydration continues for at least 1 hour with stirring at 300 rpm at 55-60° C. to form large liposomes in the range of nm to 10 microns. Downsizing of the liposomes is performed using an LM10 Microfluidizer and applying ˜18,640 psi of pressure (setting=17,500 psi) under 35-45° C. water bath and passing the full volume of liposome solution for approximately 5-12 passes to generate small liposomes (50-100 nm). Filtration of the final liposomal intermediate using 0.22 um PES sterile filter is used to generate a homogeneous population of liposomes with approximately 40-70 nm particle size with low polydistribution (PDI˜0.2). Final filtered liposome as an intermediate can be stored at 5° C. or further used to generate final adjuvant product.

### 2) Preparation of Adjuvant Product

For further manufacturing final formulation, the intermediate is equilibrated to RT and compounded with the addition of PBS buffer and QS-21 solution in PBS buffer (i.e. QS-21 at 1.5 mg/mL) to make final adjuvant formulation. 1 hour RT shaking at 300 rpm followed by additional 24 hour RT storage (with protection from light) generates the final heterogeneous adjuvant product with particle size>300 nm, and PDI>0.5. Final product is stored at 5° C. and protected from light. The size and PDI are reproducible in three repeated preparations shown in Table 12.

TABLE 12

| Formulation Batch | | | 1 | | 2 | | 3 | |
|---|---|---|---|---|---|---|---|---|
| Intermediate | Process Step | | Size (nm) | PDI | Size (nm) | PDI | Size (nm) | PDI |
| | Rehydration | | 2673 | 0.87 | 2614 | 0.57 | 3207 | 0.11 |
| | # of passes by MF | | 12 | | | | | |
| | post-downsizing | | 6 | 0.40 | 89 | 0.37 | 67 | 0.42 |
| | post-filtration | | 56 | 0.22 | 54 | 0.21 | 57 | 0.22 |
| Final | 24 hr RT by DLS | | 576 | 0.52 | 590 | 0.49 | 706 | 0.46 |
| formulation | 24 hr RT by | Mean | 397 | NA | 380 | NA | 351 | NA |
| | Horiba | D10 | 122 | NA | 177 | NA | 106 | NA |
| | Viewsizer | D50 | 350 | NA | 342 | NA | 280 | NA |
| | | D90 | 752 | NA | 702 | NA | 714 | NA |

### Example 4: Adjuvant Formulations

The present invention provides methods for making scalable amounts of MPLA-liposomal adjuvant formulations comprising a saponin (e.g. QS-21). Exemplary MPLA-liposomal adjuvant formulations comprising a saponin (e.g. QS-21) include, but are not limited to, MPLA-liposomal formulations described in U.S. Pat. No. 10,434,167 (e.g. ALFQ) and two LiNA-2 (Liposomal Novel Adjuvant-2) adjuvant formulations described herein, namely a LiNA-2 homogeneous adjuvant formulation and a LiNA-2 heterogeneous adjuvant formulation, which may be at a 1XLiNA-2 concentration or a 2XLiNA-2 concentration. The composition of these adjuvant formulations is set forth in Table 13 below:

TABLE 13
Adjuvant formulations

| | | 1XLiNA-2** | 2XLiNA-2*** |
|---|---|---|---|
| | ALFQ* | concentration | concentration |
| DMPC | 7.0 mg | 14 ± 7 | 28 ± 14 |
| | | mg/mL | mg/mL |
| DMPG | 0.78 mg | 1.6 ± 0.8 | 3.2 ± 1.6 |
| | (0.8 mg) | mg/mL | mg/mL |
| Cholesterol | 5.4 mg | 11 ± 6 | 22 ± 11 |
| | | mg/mL | mg/mL |
| MPLA | 0.2 mg | 0.40 ± 0.20 | 0.80 ± 0.40 |
| (3D-PHAD) | | mg/mL | mg/mL |
| QS-21 | 0.1 mg | 0.2 ± 0.10 | 0.4 ± 0.20 |
| | | mg/mL | mg/mL |

*described in Alving, C. R. et al., Expert Review of Vaccines 19: 3 (2020) 279-292; and Hutter, J. N. et al., Vaccine 40 (2022) 5781-5790.
**formulation for both LiNA-2 homogeneous adjuvant formulation and LiNA-2 heterogeneous adjuvant formulation.
***formulation for both LiNA-2 homogeneous adjuvant formulation and LiNA-2 heterogeneous adjuvant formulation.

### Example 5: Immunogenicity of C. difficile Vaccine Antigens Formulated with Different LiNA-2 Adjuvants

The relative immunogenicity of C. difficile toxoid antigens formulated with aluminum hydroxide (Al(OH)₃) and different LiNA-2 adjuvant formulations (homogeneous and heterogeneous) was compared in rats. Homogeneous and heterogeneous LiNA-2 adjuvants are described herein and in Table 14. Final rat LiNA-2 adjuvant doses were prepared by diluting 1× concentration of LiNA-2 at a 1:5 dilution using PBS buffer at pH 6.2.

TABLE 14
LiNA-2 adjuvant formulation

| | LiNA-2 homogenous | LiNA-2 heterogenous |
|---|---|---|
| Components | (1X, mg/mL) | (1X, mg/mL) |
| 3D-PHAD | 0.4 | 0.4 |
| QS-21 | 0.2 | 0.2 |
| DMPC | 14 | 14 |
| DMPG | 1.58 | 1.58 |
| Cholesterol | 10.82 | 10.82 |

All in 10 mM Phosphate, 150 mM NaCl solution at pH 6.2

The toxin neutralization assay (TNA) described below was used to measure the functional cytotoxic activity of sera at multiple time points following immunization.

Wistar Han rats (10 per Group, 8-10 weeks old, Charles River Laboratories) were immunized intramuscularly (IM) according to the study design in Table 15. Group 1 received C. difficile vaccine antigens formulated with Al(OH)₃. Groups 2 and 3 received the C. difficile vaccine antigens formulated with the homogeneous and heterogeneous LiNA-2 adjuvant, respectively.

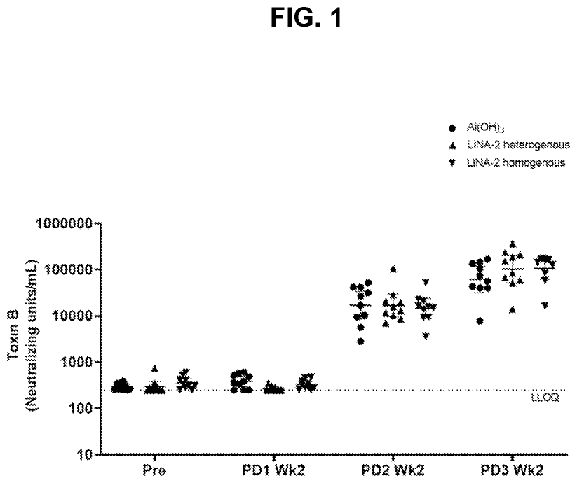

Sera were collected at multiple time points and the ability to neutralize toxin cytotoxic activity was measured in TNA's. Neutralization titers in sera from individual animals for Toxin B are shown in FIG. 1 and illustrate that toxoids formulated with homogeneous and heterogeneous LiNA-2 elicited similar immune responses able to neutralize Toxin B cytotoxicity.

TABLE 15
Rat study design with LiNA-2 adjuvanted formulations

| | | | Dose | | |
|---|---|---|---|---|---|
| | | Composition | Vol | Dosing | Assay |
| Group | Formulation | (per dose, mg/mL) | Route | Schedule | Schedule |
| 1 | Al(OH)₃ | 0.04 mg of each Txd A&B | 250 μl | Week | Week |
| | | 0.4 mg Al(OH)₃ | IM | 0, 4, 8 | 0, 2, 6, 10 |
| 2 | LiNA-2 | 0.04 mg of each Txd A&B | 250 μl | Week | Week |
| | Homogeneous | 0.08 mg 3D-PHAD ® | IM | 0, 4, 8 | 0, 2, 6, 10 |
| | | 0.04 mg QS-21 | | | |
| | | 2.8 mg DMPC | | | |
| | | 0.316 mg DMPG | | | |
| | | 2.16 mg Cholesterol | | | |
| 3 | LiNA-2 | 0.04 mg of each Txd A&B | 250 μl | Week | Week |
| | Heterogeneous | 0.08 mg 3D-PHAD ® | IM | 0, 4, 8 | 0, 2, 6, 10 |
| | | 0.04 mg QS-21 | | | |
| | | 2.8 mg DMPC | | | |
| | | 0.316 mg DMPG | | | |
| | | 2.16 mg Cholesterol | | | |

### Toxin Neutralization Assay (TNA)

Immune response induced by administering the composition of the present invention may be determined using a toxin neutralization assay (TNA), ELISA, or more preferably, a cytotoxicity assay, such as that described in WIPO Patent Application WO/2012/143902, U.S. Pat. No. 9,187,536, and WIPO Patent Application WO/2014/060898, which are each incorporated by reference herein in their respective entireties.

A toxin neutralization assay (TNA) may be used to quantitate neutralizing antibodies to C. difficile toxin. In this assay, serial diluted serum may be incubated with a fixed amount of C. difficile toxin A or B. Test cells (e.g., Vero cells) may then then added and serum-toxin-cell mixture incubated under appropriate conditions (e.g., 37° C. for 6 days). The ability of the sera to neutralize the cytotoxic effect of the C. difficile toxin may be determined by and correlated to the viability of the cells. The assay utilizes the accumulation of acid metabolites in closed culture wells as an indication of normal cell respiration. In cells exposed to toxin, metabolism and CO₂ production is reduced; consequently, the pH rises (e.g., to 7.4 or higher) as indicated by the phenol red pH indicator in the cell culture medium. At this pH, the medium appears red. Cell controls, or cells exposed to toxin which have been neutralized by antibody, however, metabolize and produce CO₂ in normal amounts; as a result, the pH is maintained (e.g., at 7.0 or below) and at this pH, the medium appears yellow. Therefore, C. difficile toxin neutralizing antibodies correlate with the ability of the serum to neutralize the metabolic effects of C. difficile toxin on cells as evidenced by their ability to maintain a certain pH (e.g., of 7.0 or lower). The color change of the media may be measured (e.g., at 562 nm to 630 nm) using a plate reader to further calculate the antitoxin neutralizing antibody titer at 50% inhibition of the C. difficile toxin-mediated cytotoxicity. In one aspect, the composition induces a toxin neutralizing antibody titer that is at least greater than 1-fold, such as, for example, at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 32-fold, or higher in the subject after receiving a dose of the composition than a toxin neutralizing antibody titer in the subject prior to receiving said dose, when measured under identical conditions in a toxin neutralization assay.

Briefly, in this Example, a 384-well microtiter plate was seeded with IMR-90 cells serving as the target of toxin-mediated cytotoxicity. Each test serum sample was analyzed separately for the ability to neutralize Toxin A or Toxin B. Four serial dilutions of test sera were mixed with fixed concentrations of Toxin A (TcdA) or Toxin B (TcdB) for 60 minutes in a humidified incubator (37° C./5% CO₂) to allow for neutralization of the toxins to occur. All plates included a reference standard and quality controls which consisted of antitoxin antibodies of known titer to monitor assay performance. After the 60-minute incubation, the toxin-antiserum mixture was applied to the IMR-90 cell monolayers and the plates were incubated for an additional 72 hours. Viability of the IMR-90 cell monolayers was then tested using the luciferase-based CellTiter-Glo® reagent which provides a measure of ATP levels in metabolically active cells and was reported as relative luminescence units (RLU). A high ATP level indicates high cell viability and antibody mediated neutralization of TcdA or TcdB. The neutralizing antibody concentration was determined by comparing the RLU value of a test sample to the calibration curve from the antitoxin A or B reference standard using a custom Statistical Analysis System (SAS®) program. The functional antibody concentrations were expressed as arbitrary units per mL (or neutralizing units/mL) of serum. The lower limit of quantitation (LLOQ) for the TcdA and TcdB TNA assays are 75.9 and 249.7 neutralizing units/mL of serum, respectively.

1. A method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol wherein the mole percent concentration of the cholesterol in the liposome composition is greater than 50% (mol/mol),
said method comprising the steps of:
(i) dissolving the phospholipids, cholesterol and MPLA in an organic solvent to form an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) removing the organic phase of the intermediate liposome of step (iii);
(v) concentrating the intermediate liposome of step (iv); and
(vi) compounding the intermediate liposome of step (v) with a saponin to form a final adjuvant formulation having a size range of about 30-400 nm with a polydispersity of 0.05 to 0.5,
thereby producing the homogeneous adjuvant formulation.

2. The method of claim 1, wherein the saponin is selected from the group consisting of QS-7, QS-18, QS-21, or a mixture thereof.

3. The method of claim 2, wherein the saponin is QS-21.

4. The method of claim 1, wherein in step (i) the phospholipids, cholesterol and MPLA are dissolved in the organic solvent by sonication, heat or a combination thereof.

5. The method of claim 4, wherein the organic solvent is ethanol or isopropyl alcohol.

6. The method of claim 4, wherein the organic phase is heated to a temperature between 45° C. to 65° C.

7. The method of claim 1, wherein the aqueous phase comprises water or a buffer.

8. The method of claim 7, wherein the buffer comprises 10 mM phosphate at pH 6.2 containing 150 mM NaCl.

9. The method of claim 7, wherein the aqueous phase is at a temperature between 20° C. to 60° C.

10. The method of claim 1, wherein the flowrate of step (ii) is 0.5 mL/min to 400 mL/min or quick addition

11. The method of claim 1, wherein the intermediate liposome of step (iii) is stirred at a rate of 700 rpm to 900 rpm.

12. The method of claim 1, wherein the ratio of organic phase to aqueous phase of step (ii) ranges from 1:4 to 1:16.

13. The method of claim 1, wherein the size of the intermediate liposome in step (iv) is downsized by using a high pressure extruder or a microfluid homogenizer.

14. The method of claim 13, wherein the size of the liposome is downsized in step (iv) by using membrane sizes ranging from 50 nm to 120 nm or homogenization pressures between 17000 PSI and 24000 PSI, or a combination of both.

15. The method of claim 1, wherein the organic solvent is removed before downsizing of step (iv) or after downsizing of step (iv).

16. The method of claim 1, wherein removing the organic phase of the intermediate liposome of step (iv) is by Tangential Flow Filtration (TFF).

17. The method of claim 16, wherein the TFF is TFF diafiltration.

18. The method of claim 16, wherein the TFF comprises membranes having a molecular weight cut-off (MWCO) ranging from 100-500 kDa.

19. The method of claim 1, wherein the concentrating of step (v) is by Ultrafiltration.

20. The method of claim 19, wherein the Ultrafiltration comprises a bioburden reduction filter and a sterile filter.

21. The method of claim 1, wherein the compounding of step (vi) is at a mixing speed of 300 rpm.

22. The method of claim 1, wherein the compounding of step (vi) ranges from 1 hour to 24 hours.

23. The method of claim 1, wherein the compounding of step (vi) occurs at room temperature or from 2-8° C.

24. The method of claim 1, wherein the final adjuvant formulation has a size range of about 30-400 nm.

25. The method of claim 1, wherein the final adjuvant formulation has a polydispersity of 0.05 to 0.5.

26. The method of claim 1, wherein the injecting of step (ii) is by pump or syringe injection.

27. A method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol wherein the mole percent concentration of the cholesterol in the liposome composition is greater than 50% (mol/mol),
said method comprising the steps of:
(i) dissolving the phospholipids, cholesterol and MPLA in an organic solvent or a mixture of organic solvents to form an organic phase;
(ii) injecting the organic phase of step (i) into an aqueous phase at a specific flowrate and at a specific ratio of the organic phase to the aqueous phase to form a liposome;
(iii) concentrating the intermediate liposome of step (ii);
(iv) removing the organic phase of the intermediate liposome of step (iii);
(v) filtering the intermediate liposome of step (iv), and
(vi) compounding the intermediate liposome of step (v) with a saponin to form a final adjuvant formulation having a size range of about 30-400 nm with a polydispersity of 0.05 to 0.5,
thereby producing the homogeneous adjuvant formulation.

28. The method of claim 27, wherein the saponin is selected from the group consisting of QS-7, QS-18, QS-21, or a mixture thereof.

29. The method of claim 28, wherein the saponin is QS-21.

30. The method of claim 27, wherein in step (i) the phospholipids, cholesterol and MPLA are dissolved in the organic solvent by sonication, heat, stirring or a combination thereof.

31. The method of claim 30, wherein the organic solvent is ethanol or isopropyl alcohol or other organic solvents.

32. The method of claim 30, wherein the organic phase is heated to a temperature between 45° C. to 65° C. Preferably, between 50° C. to 65° C. or between 45° C. to 55° C.

33. The method of claim 27, wherein the aqueous phase comprises water or a buffer.

34. The method of claim 33, wherein the buffer comprises 10 mM phosphate at pH 6.2 containing 150 mM NaCl.

35. The method of claim 33, wherein the aqueous phase is at a temperature between 20° C. to 65° C.

36. The method of claim 27, wherein the flowrate of step (ii) is 12 mL/min.

37. The method of claim 27, wherein the intermediate liposome of step (vi) is stirred at a rate of 100 rpm to 1000 rpm.

38. The method of claim 27, wherein the ratio of organic phase to aqueous phase of step (ii) ranges from 1:2 to 1:16. In a preferred embodiment, the ratio is 4:7.

39. The method of claim 27, wherein removing the organic phase of the intermediate liposome of step (iv) is by Tangential Flow Filtration (TFF).

40. The method of claim 39, wherein the TFF is TFF diafiltration.

41. The method of claim 40, wherein the TFF comprises membranes having a molecular weight cut-off (MWCO) ranging from 100-500 kDa.

42. The method of claim 27, wherein the concentrating of step (iii) is by Ultrafiltration.

43. The method of claim 27, wherein the filtering of step (v) comprises a bioburden reduction filter and a sterile filter.

44. The method of claim 27, wherein the compounding of step (vi) is at a mixing speed of 350 rpm for 1 hour at RT.

45. The method of claim 27, wherein the final adjuvant formulation has a size range of about 50-200 nm.

46. The method of claim 27, wherein the final adjuvant formulation has a polydispersity of 0.05 to 0.5.

47. The method of claim 27, wherein the injecting of step (ii) is by pump or syringe injection.

48. A method for producing a heterogeneous adjuvant formulation comprising a liposome bilayer comprising (a) monophosphoryl lipid A (MPLA), (b) a saponin, and (c) a liposome composition comprising (i) at least one phospholipid selected from at least one phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol wherein the mole percent concentration of the cholesterol in the liposome composition is greater than 50% (mol/mol),
said method comprising the steps of:
(i) dissolving the phospholipids, cholesterol and MPLA in an organic solvent to form a lipid solution as an organic phase;
(ii) simultaneously injecting the organic phase of step (i) and an aqueous phase together at specific flowrates and at a specific ratio of the organic solvent phase to the aqueous phase to form a liposome;
(iii) concentrating the intermediate liposome of step (ii);
(iv) removing the organic phase of the intermediate liposome of step (iii);
(v) treating the intermediate liposome of step (iv) by microfluidizer for 10 passes at ˜17500 psi;
(vi) filtering the intermediate liposome of step (v) using 0.22 um membrane; and
(vii) compounding the intermediate liposome of step (vi) with a saponin,
thereby producing the heterogeneous adjuvant formulation.

49. The method of claim 48, wherein the saponin is selected from the group consisting of QS-7, QS-18, QS-21, or a mixture thereof.

50. The method of claim 49, wherein the saponin is QS-21.

51. The method of claim 48, wherein in step (i) the phospholipids, cholesterol and MPLA are dissolved in the organic solvent by sonication, heat, stirring or a combination thereof.

52. The method of claim 51, wherein the organic solvent is ethanol or isopropyl alcohol or their mixture.

53. The method of claim 51, wherein the lipid formulation organic phase is heated to a temperature of 45-65° C.

54. The method of claim 48, wherein the aqueous phase comprises water or a buffer.

55. The method of claim 54, wherein the buffer comprises 10 mM phosphate at pH 6.2 containing 150 mM NaCl.

56. The method of claim 54, wherein the aqueous phase is at a temperature between 45-60° C.

57. The method of claim 48, wherein in step (ii) the organic phase has a flowrate of 1.333 ml/min and the aqueous phase has a flowrate of 10.667 ml/min (organic:aqueous=1:8, two syringes used).

58. The method of claim 48, wherein the intermediate liposome of step (iii) is stirred at a rate of 100 rpm to 900 rpm.

59. The method of claim 48, wherein the ratio of organic phase to aqueous phase of step (ii) ranges from 1:4 to 1:8.

60. The method of claim 48, wherein removing the organic phase of the intermediate liposome of step (iii) and step (iv) is by Tangential Flow Filtration (TFF).

61. The method of claim 60, wherein the size of the intermediate liposome in step (iv) is treated by using a microfluidizer).

62. The method of claim 48, wherein the organic solvent is removed before microfluidizing of step (v)

63. The method of claim 60, wherein the TFF is TFF ultrafiltration and diafiltration.

64. The method of claim 60, wherein the TFF comprises membranes having a molecular weight cut-off (MWCO) ranging from 100-500 kDa.

65. The method of claim 48, wherein the concentrating of step iii) is by Ultrafiltration.

66. The method of claim 48, wherein a buffer is added to the compounding of step (vi).

67. The method of claim 48, wherein the compounding of step (vii) is at a mixing speed of 350 rpm for 1 hour to 48 at RT or agitated for 1 hr.

68. The of claim 67, wherein following the compounding of step (vii) the intermediate liposome is stored at RT without stirring for up to 48 hours.

69. The method of claim 48, wherein the final adjuvant formulation has a size of about 300 to 1000 nm.

70. The method of claim 48, wherein the final adjuvant formulation has a polydispersity of 0.4-1.

71. The method of claim 48, wherein the injecting of step (ii) is by Nanoassemblr or pump or syringe injection.

72. A method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol wherein the mole percent concentration of the cholesterol in the liposome composition is greater than 50% (mol/mol),
said method comprising the steps of:
(i) dissolving the phospholipids, cholesterol and MPLA in an organic solvent to form a lipid formulation an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) concentrating the intermediate liposome of step (iii);
(v) removing the organic phase of the intermediate liposome of step (iv); and
(vi) compounding the intermediate liposome of step (vi) with a saponin,
thereby producing the heterogeneous adjuvant formulation.

73. The method of claim 72, wherein the saponin is selected from the group consisting of QS-7, QS-18, QS-21, or a mixture thereof.

74. The method of claim 73, wherein the saponin is QS-21.

75. The method of claim 72, wherein in step (i) the phospholipids, cholesterol and MPLA are dissolved in the organic solvent by sonication, heat, stirring or a combination thereof.

76. The method of claim 75, wherein the organic solvent comprises ethyl acetate and isopropyl alcohol.

77. The method of claim 75, wherein the lipid formulation organic phase is heated to a temperature from 50° C. to 65° C.

78. The method of claim 72, wherein the aqueous phase comprises water or a buffer.

79. The method of claim 78, wherein the buffer comprises 10 mM phosphate at pH 6.2 containing 150 mM NaCl.

80. The method of claim 78, wherein the aqueous phase is at a temperature of 20-25° C.

81. The method of claim 72, wherein the flowrate of step (ii) is 20 mL/min.

82. The method of claim 72, wherein the intermediate liposome of step (iii) is stirred at a rate of 100 rpm to 900 rpm.

83. The method of claim 72, wherein the ratio of organic phase to aqueous phase of step (ii) ranges from 1:4 to 1:8.

84. The method of claim 72, wherein the organic solvent is removed before compounding of step (vi).

85. The method of claim 72, wherein removing the organic phase of the intermediate liposome of step (iv) and step (v) is by Tangential Flow Filtration (TFF).

86. The method of claim 85, wherein the TFF is TFF ultrafiltration and diafiltration.

87. The method of claim 85, wherein the TFF comprises membranes having a molecular weight cut-off (MWCO) ranging from 100-500 kDa.

88. The method of claim 72, wherein the concentrating of step (vi) is by Ultrafiltration.

89. The method of claim 72, wherein a buffer is added to the compounding of step (vi).

90. The method of claim 72, wherein the compounding of step (vi) is at a mixing speed of 300 rpm for 1 hour at RT.

91. The of claim 90, wherein following the compounding of step (vii) the intermediate liposome is stored at RT without stirring for 24 hours.

92. The method of claim 72, wherein the final adjuvant formulation has a size range of 300 nm to 1000 nm.

93. The method of claim 72, wherein the final adjuvant formulation has a polydispersity of 0.4 to 1.0.

94. The method of claim 72, wherein the injecting of step (ii) is by pipette, pump or syringe injection.

95. A method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol wherein the mole percent concentration of the cholesterol in the liposome composition is greater than 50% (mol/mol),
said method comprising the steps of:
(i) preparing a lyopilized organic phase comprising the phospholipids, cholesterol and MPLA;
(ii) rehydrating the lyophilized organic phase of step (i) with an aqueous phase to form an intermediate liposome;
(iii) Downsizing of the intermediate liposome of step (ii) using microfluidizer;
(iv) compounding the intermediate liposome of step (iii) with a saponin,
thereby producing the heterogeneous adjuvant formulation.

96. The method of claim 95, wherein the saponin is selected from the group consisting of QS-7, QS-18, QS-21, or a mixture thereof.

97. The method of claim 96, wherein the saponin is QS-21.

98. The method of claim 95, wherein in step (i) the phospholipids, cholesterol and MPLA are dissolved in the organic solvent by sonication, heat, stirring or a combination thereof.

99. The method of claim 98, wherein the organic solvent comprises tert-butyl alcohol (TBA) or its mixture.

100. The method of claim 99, wherein the lipid organic phase is heated to a temperature from 25° C. to 65° C.

101. The method of claim 100, wherein the organic phase solution is lyophilized.

102. The method of claim 95, wherein the aqueous phase comprises water or a buffer.

103. The method of claim 102, wherein the buffer comprises 10 mM phosphate at pH 6.2 containing 150 mM NaCl.

104. The method of claim 102, wherein the aqueous phase is at a temperature from 20° C. to 70° C.

105. The method of claim 95, wherein the intermediate liposome of step (ii) is stirred at a rate of 100-1000 rpm.

106. The method of claim 95, wherein the size of the intermediate liposome in step (iii) is downsized with a microfluidizer and pressure of 18640 PSI.

107. The method of claim 95, wherein the organic solvent is removed before rehydration of step (ii).

108. The method of claim 95, wherein removing the organic solvent by lyophilization.

109. The method of claim 95, wherein a buffer is added to the compounding of step (iv).

110. The method of claim 95, wherein the compounding of step (iv) is at a mixing speed of 300 rpm or by agitation for 1 hour at RT.

111. The of claim 110, wherein following the compounding of step (iv) the intermediate liposome is stored at RT without stirring for 24 hours.

112. The method of claim 95, wherein the final adjuvant formulation has a size>300 nm.

113. The method of claim 95, wherein the final adjuvant formulation has a polydispersity>0.4

114. An adjuvant formulation produced by any one of the methods according to claims 1-113.
